(19) United States
(12) Patent Application Publication
(10) Pub. No.: US 2012/0329071 A1
(43) Pub. Date: Dec. 27, 2012

(54) PROTEIN BIOMARKERS AND THERAPEUTIC TARGETS FOR RENAL DISORDERS

(76) Inventors: Mark Chance, Moreland Hills, OH (US); Daniella Schlatzer, Lakewood, OH (US)

(21) Appl. No.: 13/581,939

(22) PCT Filed: Mar. 7, 2011
(86) PCT No.: PCT/US2011/027440

§ 371 Date: Aug. 30, 2012

Related U.S. Application Data
(60) Provisional application No. 61/310,842, filed on Mar. 5, 2010.

(51) Int. Cl. G01N 33/566; G01N 33/573

(52) U.S. Cl. 435/7.4; 435/7.92

(57) ABSTRACT

The present invention relates to a method of diagnosing a renal disorder. The method includes the steps of: (1) obtaining a biological sample from a subject; and (2) determining, in the biological sample, a level of one or more proteins whose abundance in urine change due to the renal disorder, wherein an increase or decrease in the level of one or more of the proteins compared to a control level is indicative of a renal disorder.

### RELATED APPLICATION

This application claims priority from U.S. Provisional Application Nos. 61/310,842, filed Mar. 5, 2010, the subject matter of which are incorporated herein by reference in their entirety.

### BACKGROUND

Diabetes mellitus (DM) is estimated to affect approximately 24 million people in the United States alone. There are numerous end organ complications of diabetes, such as nephropathy, retinopathy, cardiovascular disease, and bladder dysfunction. There are also multiple risk factors for these complications; they include level of glycemic control, blood pressure increases, obesity, and family history. Also, African Americans and other ethnic groups are at in increased risk. The development of molecular markers to better understand and stratify risk for specific patient populations is an important goal in improving health care and developing better preventative medicine strategies. Multiple effective therapies to reduce the problems of diabetes exist. However, early diagnosis and intervention are essential to properly manage the progression of complications, in particular, coronary artery disease and end stage renal disease.

As an example, the complication of diabetic nephropathy accounts for approximately 44% of new cases of end stage renal disease (ESRD or chronic kidney disease, CKD). In addition, coronary artery disease (CAD) is a well-known co-morbidity for renal disease and is a well-known and increasingly prevalent complication of diabetes. The increases in these renal and cardiovascular complications are attributed to increases in prevalence of both Type 2 diabetes and decrease of mortality of patients with Type 1 or Type 2 DM as well as increases in hypertension. As higher numbers of patients live longer, the incidence of diabetic complications such as nephropathy and CAD increase. Moreover, Type 1 DM patients who progress to ESRD have an increased risk of mortality with estimated cost of Type 1 DM related ESRD in the United States to be approximately $1.9 billion.

Two keystone therapies for the prevention and management of ESRD are aggressive glycemic control and blood pressure regulation. Early intervention is essential in reducing the severity and course of this complication. Determination of urinary total protein (UTP), serum creatine, and microalbuminuria are commonly used measurements to monitor and predict renal complications and cardiovascular risk. Although these assays are the gold standard for diagnosing risk for Type 1 and are also likely appropriate for diagnosing risk for Type 2 DM patients, these assays lack both sensitivity and specificity that results in under-representing at risk DM patients. Therefore, new assays are required to accurately target these patients for therapeutic intervention before the onset of disease. Serum cystatin-C is one such protential marker, but the level of improvement over the gold standard assay is statistically significant but small.

### BRIEF DESCRIPTION OF DRAWINGS

FIG. 1 is a flow diagram illustrating a method for detecting diabetes in a subject in accordance with one aspect of the present invention.

FIG. 2 is a flow diagram illustrating a method for treating diabetes in a subject in accordance with one aspect of the present invention.

FIG. 3 illustrates a graph showing classification performance of the label free progression predictive model by final MA.

FIG. 4 illustrates the levels of proteins by ELISA by progression to albluminuria and diabetes status.

### SUMMARY OF THE INVENTION

The present invention relates to the use of protein expression profiles for the detection of renal disorders and the detection of diabetes. In particular, the invention identifies proteins whose abundance levels in a biological sample can be correlated with diabetes or end stage renal disease (ESRD). These protein expression profiles may be used for the diagnosis of diabetes, such as type 1 diabetes or ESRD. Compared to existing methods of diagnosis, the protein expression profiles disclosed herein constitute a reliable and consistent urinary protein profiles in diabetic patients, and provide a more reliable basis for the selection of appropriate therapeutic regimens.

An aspect of the application involves the use of expression profiles of the marker proteins listed in Table 1 in a method for diagnosing whether a subject has an increased risk of a renal disorder or diabetes (e.g., type 1 diabetes). The method includes: (1) obtaining a biological sample from a subject; and (2) determining, in the biological sample, a level of one or more of the polypeptide selected from the group consisting of the proteins recited in Table 1 (i.e, Isoform 1 of Uromodulin, Serotransferrin precursor, Isoform Long of Trifunctional purine biosynthetic protein adenosine-3, Cystatin-A, alpha-2-glycoprotein 1 zinc, ubiquitin and ribosomal protein S27a precursor, C16orf73 Novel protein, Zinc finger homeobox protein 2, Muscarinic acetylcholine receptor M1, WDR2OWD repeat domain 20 isoform 3, Interleukin 20 receptor alpha, Isoform 4 of VPS 10 domain-containing receptor SorCS 1 precursor, CDNA FLI41726 fis clone HLUNG2014449, Similar to CF3558-PA isoform A, Solute carrier family 16 member 2, trefoil factor 3 precursor, RNASE2 Nonsecretory ribonuclease precursor, Secreted Ly-6/upar-related protein 1 precursor, RGSL1 Hypothetical Protein, Isoform LMW of Kininogen-1 precursor, Isoform 3 of Killer cell immunoglobulin-like receptor 2DL4 precursor, Melanoma-associated antigen B6, isoform A of AP-2 complex subunit alpha-1, secreted phosphoprotein 1 isoform b, Cytochrome P450 4F11, Insulin growth factor-like family member 4 precursor, SLC39A7 Non-class II protein, C19orf57 Uncharacterized protein, SERPINA Alpha-1-antitrypsin precursor, ALB Isoform 1 of Serum albumin precursor, Mucin-5B precursor, analogs and fragments thereof) wherein an increase or decrease in the level of one or more of the proteins compared to a control level is indicative of the subject having a renal disorder or an increased risk of a renal disorder.

Another aspect of the application relates to a method of diagnosing an increased risk type 1 diabetes in a subject. The method includes: (1) obtaining a biological sample from a subject; and (2) determining, in the biological sample, the level of one or more of polypeptides selected from the group consisting of the proteins listed in Table 1 (i.e., Isoform 1 of Uromodulin, Serotransferrin precursor, Isoform Long of Trifunctional purine biosynthetic protein adenosine-3, Cystatin-A, alpha-2-glycoprotein 1 zinc, ubiquitin and ribosomal protein S27a precursor, C16orf73 Novel protein, Zinc finger homeobox protein 2, Muscarinic acetylcholine receptor M1, WDR2OWD repeat domain 20 isoform 3, Interleukin 20 receptor alpha, Isoform 4 of VPS10 domain-containing receptor SorCS1 precursor, CDNA FLI41726 fis clone HLUNG2014449, Similar to CF3558-PA isoform A, Solute carrier family 16 member 2, trefoil factor 3 precursor, RNASE2 Nonsecretory ribonuclease precursor, Secreted Ly-6/upar-related protein 1 precursor, RGSL1 Hypothetical Protein, Isoform LMW of Kininogen-1 precursor, Isoform 3 of Killer cell immunoglobulin-like receptor 2DL4 precursor, Melanoma-associated antigen B6, isoform A of AP-2 complex subunit alpha-1, secreted phosphoprotein 1 isoform b, Cytochrome P450 4F11, Insulin growth factor-like family member 4 precursor, SLC39A7 Non-class II protein, C19orf57 Uncharacterized protein, SERPINA Alpha-1-antitrypsin precursor, ALB Isoform 1 of Serum albumin precursor, Mucin-5B precursor, analogs and fragments thereof), wherein an increase or decrease in the level of the proteins identified in Table 1 compared to a control level is indicative of the subject having diabetes or increased risk of having diabetes.

The application further relates to the use of expression profiles of the marker proteins listed in Table 2 in a method for diagnosing a sub-type of type-1 diabetes disease responsive to treatment by an angiotensin-converting enzyme inhibitor and/or an angiotensin receptor blocker (e.g., type-1 diabetes with microalbuminuria). The method includes: (1) obtaining a biological sample from a subject; and (2) determining, in the biological sample, the level of one or more of polypeptides selected from the group consisting of the proteins presented in Table 2 (i.e., Apolipoprotein D precursor, APOH Beta-2-glycoprotein 1 precursor, CD59 glycoprotein 1 precursor, CD99 Isoform II of CD99 antigen precursor, CD99L2 protein DKFZp761H2O24, CLU, Collagen alpha-1(1) chain precursor, Cystatin-A, Beta-defensin 1 precursor, Isoform 2 of Granulins precursor, Basement membrane-specific heparin sulfate proteoglycan core protein precursor, IGKC protein, Inter-alpha-trypsin inhibitor heavy chain H2 precursor, Isoform LMW of Kininogen-1 precursor, Isoform 1 of Peptidase inhibitor 16 precursor, Polymeric-immunoglobulin receptor precursor, Isoform 2 of Phosphoinositide-3-kinase-interacting protein 1 precursor, isoform Sap-mu-0 of Proactivator polypeptide precursor, Prostaglandin-H2 D-isomerase precursor, Transcriptional activator protein Pur-Alpha, RNASE1 Ribonuclease pancreatic precursor, RNASE2 Nonsecretory ribonuclease precursor, RPS27A;UBC;UBB ubiquitin and ribosomal protein S27a precursor, Secreted Ly-6/uPar-related protein 1 precursor, secreted phosphoprotein 1 isoform b, Trefoil factor 2 precursor, Isoform 1 of Uromodulin precursor, VGF nerve growth factor inducible precursor, Isoform 1 of WAP four-disulfide core domain protein 2 precursor, AMBP protein precursor, Annexin A1, alpha-2-glycoprotein 1 zinc, Beta-2-microglobulin precursor, C gamma 3, Ceruloplasmin precursor, Hemopexin precursor, Mucin-5B precursor, ORM2 Alpha-1-acid glycoprotein 2 precursor, SERPINA1 Alpha-1-antitrypsin precursor, TF Serotransferrin precursor, analogs and fragments thereof), wherein an increase or decrease in the level of one or more of the proteins compared to a control level is indicative of a sub-type of type 1 diabetes responsive to treatment by an angiotensin-converting enzyme inhibitor and/or an angiotensin receptor blocker.

Another aspect of the application relates to a method of treating type 1 diabetes in a subject. The method includes: (1) obtaining a biological sample from a subject; and (2) determining, in the biological sample, a level of one or more of polypeptides selected from the group consisting of the proteins presented in Table 2 (i.e., Apolipoprotein D precursor, APOH Beta-2-glycoprotein 1 precursor, CD59 glycoprotein 1 precursor, CD99 Isoform II of CD99 antigen precursor, CD99L2 protein DKFZp761H2O24, CLU, Collagen alpha-1(1) chain precursor, Cystatin-A, Beta-defensin 1 precursor, Isoform 2 of Granulins precursor, Basement membrane-specific heparin sulfate proteoglycan core protein precursor, IGKC protein, Inter-alpha-trypsin inhibitor heavy chain H2 precursor, Isoform LMW of Kininogen-1 precursor, Isoform 1 of Peptidase inhibitor 16 precursor, Polymeric-immunoglobulin receptor precursor, Isoform 2 of Phosphoinositide-3-kinase-interacting protein 1 precursor, isoform Sap-mu-0 of Proactivator polypeptide precursor, Prostaglandin-H2 D-isomerase precursor, Transcriptional activator protein Pur-Alpha, RNASE1 Ribonuclease pancreatic precursor, RNASE2 Nonsecretory ribonuclease precursor, RPS27A;UBC;UBB ubiquitin and ribosomal protein S27a precursor, Secreted Ly-6/uPar-related protein 1 precursor, secreted phosphoprotein 1 isoform b, Trefoil factor 2 precursor, Isoform 1 of Uromodulin precursor, VGF nerve growth factor inducible precursor, Isoform 1 of WAP four-disulfide core domain protein 2 precursor, AMBP protein precursor, Annexin Al, alpha-2-glycoprotein 1 zinc, Beta-2-microglobulin precursor, C gamma 3, Ceruloplasmin precursor, Hemopexin precursor, Mucin-5B precursor, ORM2 Alpha-1-acid glycoprotein 2 precursor, SERPINA1 Alpha-1-antitrypsin precursor, TF Serotransferrin precursor, analogs and fragments thereof), wherein an increase or decrease in the level of one or more of the proteins compared to a control level is indicative of a sub-type of type 1 diabetes responsive to treatment by an angiotensin-converting enzyme inhibitor and/or an angiotensin receptor blocker; and (3) administering an angiotensin-converting enzyme inhibitor and/or an angiotensin receptor blocker to the subject.

The application further relates to the use of expression profiles of the marker proteins listed in Table 4 in a method for diagnosing an increased risk of development of renal function decline and/or end-stage renal disease (ESRD) in a subject with type-1 diabetes. The method includes: (1) obtaining a biological sample from a subject; and (2) determining, in the biological sample, the level of one or more of polypeptides selected from the group consisting of the proteins presented in Table 4 (i.e., Isofrom 1 of Serum albumin, Protein AMBP, Apolipoprotein D, alpha-2-glycoprotein 1, zinc, CD59 glycoprotein, Putative uncharacterized protein CD99, cDNA FLJ57622, highly similar to Clusterin, Ceruloplasmin, Isoform 1 of Fibrinogen alpha chain, Basement membrane-specific heparin sulfate proteoglycan core protein, IGHM protein, IGLV4-3 protein, Isoform LMW of Kininogen-1, Keratin, type II cytoskeletal 1, Keratin, type I cytoskeletal 9, Matrix Gla protein, cDNA FLJ59142, highly similar to Epididymal secretory protein E1, Alpha-1-acid glycoproteinl, Prolactin-inducible protein, Prosaposin, Prostaglandin-H2 D-isomerase, Isoform 1 of Semenogelin-1, Semenogelin-2, Isoform 1 of Alpha-1-antitryspin, Antithrombin III variant, Plasma protease C1 inhibitor, Anchor protein, Secreted Ly-6/uPAR-related protein 1, Protein TET1, Serotransferrin, Isoform 1 of Uromodulin, Neurosecretory VGF, precursor, analogs and fragments thereof), wherein an increase or decrease in the level of one or more of the proteins compared to a control level is indicative of the subject with type 1 diabetes having an increased risk of renal function decline and/or ESRD.

A further aspect of the application relates to the use of expression profiles of the marker proteins listed in Table 5 in a method for diagnosing an increased risk of development of renal function decline and/or end-stage renal disease ESRD in a subject. The method includes: (1) obtaining a biological sample from a subject; and (2) determining, in the biological sample, the level of one or more of polypeptides selected from the group consisting of Tamms Horsfall Protein (THP), progranulin, and alpha-1-acid glycoprotein (AGP), wherein an increase or decrease in the level of one or more of the proteins compared to a control level is indicative of the subject having an increased risk of renal function decline, and/or ESRD.

In the methods of the application, the biological sample can include a biological fluid sample (e.g., a sample of urine). In certain preferred embodiments, the biological sample includes a sample of urine.

In certain aspects of the application, the subject is a human being, for example, a subject suspected of having a renal disorder.

### DETAILED DESCRIPTION

Throughout the specification, several terms are employed that are defined in the following paragraphs.

As used herein, the term “subject” and “individual” are used herein interchangeably. They refer to a human or another mammal (e.g., primate, dog, cat, goat, horse, pig, mouse, rat, rabbit, and the like), that can be afflicted with diabetes, such as diabetes but may or may not have the disease. In many embodiments, the subject is a human being.

As used herein, the term “diagnosis” refers to a process aimed at determining if an individual is afflicted with a disease or ailment. In the context of the present invention, “diagnosis of diabetes” refers to a process aimed at one or more of: determining if a subject is likely to develop diabetes, determining if a subject is afflicted with diabetes.

As used herein, the term “biological sample” is used herein in its broadest sense. A biological sample may be obtained from a subject (e.g., a human) or from components (e.g., tissues) of a subject. The sample may be of any biological tissue or fluid with which biomarkers of the present invention may be assayed. Frequently, the sample will be a “clinical sample”, i.e., a sample derived from a patient. Such samples include, but are not limited to, bodily fluids, e.g., urine, blood, blood plasma, saliva; tissue or fine needle biopsy samples; and archival samples with known diagnosis, treatment and/or outcome history. The term biological sample also encompasses any material derived by processing the biological sample. Derived materials include, but are not limited to, cells (or their progeny) isolated from the sample, proteins or nucleic acid molecules extracted from the sample. Processing of the biological sample may involve one or more of, filtration, distillation, extraction, concentration, inactivation of interfering components, addition of reagents, and the like.

As used herein, the terms “normal” and “healthy” are used herein interchangeably. They refer to an individual or group of individuals who have not shown any renal disorder symptoms, such as diabetes, and have not been diagnosed with diabetes. Preferably, said normal individual (or group of individuals) is not on medication affecting diabetes. In certain embodiments, normal individuals have similar sex, age, body mass index as compared with the individual from which the sample to be tested was obtained. The term “normal” is also used herein to qualify a sample isolated from a healthy individual.

As used herein, the term “control sample” refers to one or more biological samples isolated from an individual or group of individuals that are normal (i.e., healthy). The term “control sample” (or “control”) can also refer to the compilation of data derived from samples of one or more individuals classified as normal, or one or more individuals diagnosed with diabetes.

As used herein, the terms “biomarker” refers to a protein selected from the set of proteins provided by the present invention and whose expression profile was found to be indicative of diabetes, such as type 1 diabetes, or end-stage renal disease (ESRD). The term “biomarker” also encompasses nucleic acid molecules comprising a nucleotide sequence, which codes for a marker protein of the present invention as well as polynucleotides that hybridize with portions of these nucleic acid molecules.

As used herein, the term “indicative of diabetes”, when applied to a biomarker, refers to an expression pattern or profile, which is diagnostic of diabetes such that the expression pattern is found significantly more often in subjects with the disease than in patients without the disease or another subtype of the disease (as determined using routine statistical methods setting confidence levels at a minimum of 95%). Preferably, an expression pattern, which is indicative of diabetes is found in at least 60% of patients who have the disease and is found in less than 10% of subjects who do not have the disease. More preferably, an expression pattern which is indicative of diabetes is found in at least 70%, at least 75%, at least 80%, at least 85%, at least 90%, at least 95% or more in patients who have the disease and is found in less than 10%, less than 8%, less than 5%, less than 2.5%, or less than 1% of subjects who do not have the disease.

As used herein, the term “differentially expressed biomarker” refers to a biomarker whose abundance level is different in a subject (or a population of subjects) afflicted with diabetes relative to its level in a healthy or normal subject (or a population of healthy or normal subjects). Differential expression includes quantitative, as well as qualitative, differences in the temporal or cellular expression pattern of the biomarker. As described in greater details below, a differentially expressed biomarker, alone or in combination with other differentially expressed biomarkers, is useful in a variety of different applications in diagnostic, sub-typing, therapeutic, drug development and related areas. The expression patterns of the differentially expressed biomarkers disclosed herein can be described as a fingerprint or a signature of diabetes, diabetes subtype and diabetes progression. They can be used as a point of reference to compare and characterize unknown samples and samples for which further information is sought. The term “decreased level” as used herein, refers to a decrease in the abundance level of one or more of the biomarkers described herein of at least 10% or more. For example, 20%, 30%, 40%, or 50%, 60%, 70%, 80%, 90% or more, or a decrease of greater than 1-fold, 2-fold, 3-fold, 4-fold, 5-fold, 10-fold, 50-fold, 100-fold or more as measured by one or more methods described herein. The term “increased level” as used herein, refers to an increase in the abundance one or more of the biomarkers described herein of at least 10% or more. For example, 20%, 30%, 40%, or 50%, 60%, 70%, 80%, 90% or more, or an increase of greater than 1-fold, 2-fold, 3-fold, 4-fold, 5-fold, 10-fold, 50-fold, 100-fold or more as measured by one or more methods, such as method described herein.

As used herein, the terms “protein”, “polypeptide”, and “peptide” are used herein interchangeably, and refer to amino acid sequences of a variety of lengths, either in their neutral (uncharged) forms or as salts, and either unmodified or modified by glycosylation, side chain oxidation, or phosphorylation. In certain embodiments, the amino acid sequence is the full-length native protein. In other embodiments, the amino acid sequence is a smaller fragment of the full-length protein. In still other embodiments, the amino acid sequence is modified by additional substituents attached to the amino acid side chains, such as glycosyl units, lipids, or inorganic ions such as phosphates, as well as modifications relating to chemical conversion of the chains, such as oxidation of sulfhydryl groups. Thus, the term “protein” (or its equivalent terms) is intended to include the amino acid sequence of the full-length native protein, subject to those modifications that do not change its specific properties. In particular, the term “protein” encompasses protein isoforms, i.e., variants that are encoded by the same gene, but that differ in their pI or MW, or both. Such isoforms can differ in their amino acid sequence (e.g., as a result of alternative splicing or limited proteolysis), or in the alternative, may arise from differential post-translational modification (e.g., glycosylation, acylation, phosphorylation).

As used herein, the term “protein analog”, as used herein, refers to a polypeptide that possesses a similar or identical function as the full-length native protein but need not necessarily comprise an amino acid sequence that is similar or identical to the amino acid sequence of the protein, or possesses a structure that is similar or identical to that of the protein. Preferably, in the context of the present invention, a protein analog has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the full-length native protein.

As used herein, the term “protein fragment”, as used herein, refers to a polypeptide comprising an amino acid sequence of at least 4 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino acid residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a second polypeptide. The fragment of a marker protein may or may not possess a functional activity of the full-length native protein.

As used herein, the terms “nucleic acid molecule” and “polynucleotide” are used herein interchangeably. They refer to a deoxyribonucleotide or ribonucleotide polymer in either single- or double-stranded form, and unless otherwise stated, encompass known analogs of natural nucleotides that can function in a similar manner as naturally occurring nucleotides. The terms encompass nucleic acid-like structures with synthetic backbones, as well as amplification products.

As used herein, the term “a reagent that specifically detects levels” refers to one or more reagents used to detect the level of one or more biomarkers (e.g., a polypeptide selected from the marker proteins provided herein, a nucleic acid molecule comprising a polynucleotide sequence coding for a marker protein, or a polynucleotide that hybridizes with at least a portion of the nucleic acid molecule). Examples of suitable reagents include, but are not limited to, antibodies capable of specifically binding to a marker protein of interest, nucleic acid probes capable of specifically hybridizing to a polynucleotide sequence of interest, or PCR primers capable of specifically amplifying a polynucleotide sequence of interest. The term “amplify” is used herein in the broad sense to mean creating/generating an amplification product. “Amplification”, as used herein, generally refers to the process of producing multiple copies of a desired sequence, particularly those of a sample. A “copy” does not necessarily mean perfect sequence complementarity or identity to the template sequence.

As used herein, the term “hybridizing” refers to the binding of two single stranded nucleic acids via complementary base pairing. The term “specific hybridization” refers to a process in which a nucleic acid molecule preferentially binds, duplexes, or hybridizes to a particular nucleic acid sequence under stringent conditions (e.g., in the presence of competitor nucleic acids with a lower degree of complementarity to the hybridizing strand). In certain embodiments of the present invention, these terms more specifically refer to a process in which a nucleic acid fragment (or segment) from a test sample preferentially binds to a particular probe and to a lesser extent or not at all, to other probes, for example, when these probes are immobilized on an array.

As used herein, the terms “array”, “micro-array”, and “biochip” are used herein interchangeably. They refer to an arrangement, on a substrate surface, of hybridizable array elements, preferably, multiple nucleic acid molecules of known sequences. Each nucleic acid molecule is immobilized to a discrete spot (i.e., a defined location or assigned position) on the substrate surface. The term “micro-array” more specifically refers to an array that is miniaturized so as to require microscopic examination for visual evaluation.

As used herein, the term “probe”, as used herein, refers to a nucleic acid molecule of known sequence, which can be a short DNA sequence (i.e., an oligonucleotide), a PCR product, or mRNA isolate. Probes are specific DNA sequences to which nucleic acid fragments from a test sample are hybridized. Probes specifically bind to nucleic acids of complementary or substantially complementary sequence through one or more types of chemical bonds, usually through hydrogen bond formation.

As used herein, the terms “labeled”, “labeled with a detectable agent” and “labeled with a detectable moiety” are used herein interchangeably. These terms are used to specify that an entity (e.g., a probe) can be visualized, for example, following binding to another entity (e.g., a polynucleotide or polypeptide). Preferably, the detectable agent or moiety is selected such that it generates a signal which can be measured and whose intensity is related to the amount of bound entity. In array-based methods, the detectable agent or moiety is also preferably selected such that it generates a localized signal, thereby allowing spatial resolution of the signal from each spot on the array. Methods for labeling polypeptides or polynucleotides are well-known in the art. Labeled polypeptides or polynucleotides can be prepared by incorporation of or conjugation to a label, that is directly or indirectly detectable by spectroscopic, photochemical, biochemical, immunochemical, electrical, optical, or chemical means. Suitable detectable agents include, but are not limited to, various ligands, radionuclides, fluorescent dyes, chemiluminescent agents, microparticles, enzymes, calorimetric labels, magnetic labels, and haptens. Detectable moieties can also be biological molecules such as molecular beacons and aptamer beacons.

As used herein, the term “diabetes expression profile map” refers to a presentation of expression levels of a set of biomarkers in a particular status of diabetes (e.g., absence of disease, diabetes, diabetes responsive to treatment by an angiotensin-converting enzyme inhibitor and/or an angiotensin receptor blocker). The map may be presented as a graphical representation (e.g., on paper or a computer screen), a physical representation (e.g., a gel or array) or a digital representation stored in a computer-readable medium. Each map corresponds to a particular status of the disease (e.g., absence of disease, diabetes, diabetes responsive to treatment by an angiotensin-converting enzyme inhibitor and/or an angiotensin receptor blocker), and thus provides a template for comparison to a patient sample. In certain preferred embodiments, maps are generated from a plurality of samples obtained from a significant number of normal individuals or individuals with the same subtype of diabetes. Maps may be established for individuals with matched age, sex and body mass index.

This application relates to methods of diagnosing an increased risk of diabetes as well as an increased risk of development of renal function decline, and/or end-stage renal disease ESRD in a subject. The present application also relates to improved systems and strategies for the diagnostic, characterization, and sub-typing of diabetes as well renal function decline and/or end-stage renal disease ESRD in a subject. The application further relates to markers that can identify subjects who have type 1 diabetes and/or a sub-type of type 1 diabetes responsive to treatment by an angiotensin-converting enzyme inhibitor and/or an angiotensin receptor blocker.

The markers described herein listed in Table 1, Table 2, Table 4, and Table 5 can be used in the present methods as prognostic tools to effectively target and trigger therapeutic intervention earlier than currently described. The present application describes specific markers and assays in biological samples to detect the identified markers. These markers reflect early changes in glomerular filtration rate (GER) and biological changes in kidney that indicates progression of renal disease and disorders along with the risk of coronary artery disease (CAD) as well as an increased risk of development of renal function decline and/or end-stage renal disease ESRD in a subject. The indicated markers can be used as prognostic indicators to trigger standard and effective therapeutic interventions with angiotensin converting enzyme inhibitor (ACE-I) and/or angiotensin receptor blocker (ARB) treatments earlier than currently prescribed, likely lowering the risk of both end stage renal disease (ESRD) and CAD.

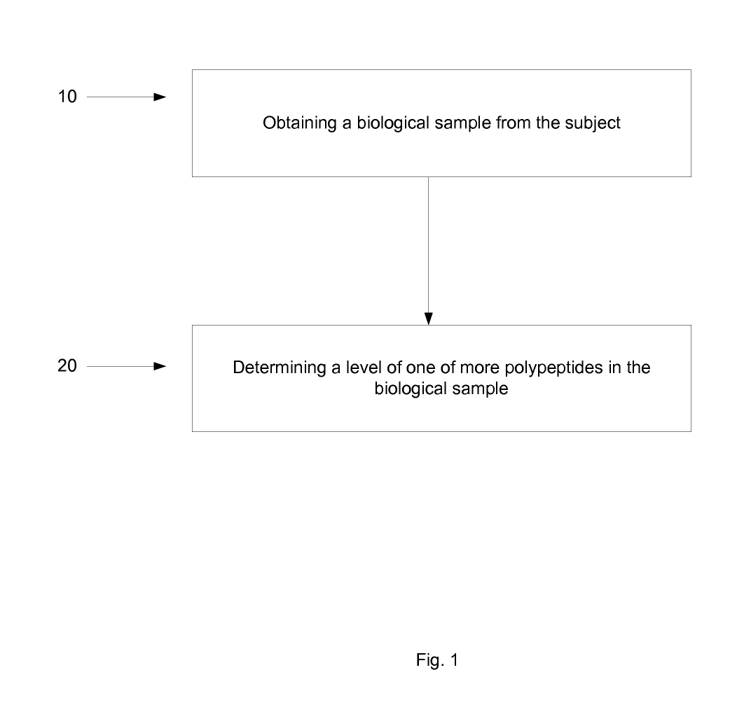

It was found that proteins whose abundance in urine changes due to diabetes can discriminate diabetic subjects from healthy controls. Therefore, a first aspect of the application provides a method for diagnosing a renal disorder or an increased risk of a renal disorder in a subject. As shown in FIG. 1, at 10, the method includes a first step of obtaining a biological sample from subject. At 20, the method also includes a second step of determining a level of one or more of the polypeptide selected from the group consisting of Isoform 1 of Uromodulin, Serotransferrin precursor, Isoform Long of Trifunctional purine biosynthetic protein adenosine-3, Cystatin-A, alpha-2-glycoprotein 1 zinc, ubiquitin and ribosomal protein S27a precursor, C16orf73 Novel protein, Zinc finger homeobox protein 2, Muscarinic acetylcholine receptor M1, WDR20WD repeat domain 20 isoform 3, Interleukin 20 receptor alpha, Isoform 4 of VPS10 domain-containing receptor SorCS1 precursor, CDNA FLI41726 fis clone HLUNG2014449, Similar to CF3558-PA isoform A, Solute carrier family 16 member 2, trefoil factor 3 precursor, RNASE2 Nonsecretory ribonuclease precursor, Secreted Ly-6/upar-related protein 1 precursor, RGSL1 Hypothetical Protein, Isoform LMW of Kininogen-1 precursor, Isoform 3 of Killer cell immunoglobulin-like receptor 2DL4 precursor, Melanoma-associated antigen B6, isoform A of AP-2 complex subunit alpha-1, secreted phosphoprotein 1 isoform b, Cytochrome P450 4F11, Insulin growth factor-like family member 4 precursor, SLC39A7 Non-class II protein, C19orf57 Uncharacterized protein, SERPINA Alpha-1-antitrypsin precursor, ALB Isoform 1 of Serum albumin precursor, and Mucin-5B precursor. The increase or decrease of the level of one or more of these proteins when compared to a control level is indicative of a renal disorder or an increased risk of a renal disorder in a subject.

The renal disorder diagnosed using the methods of the present invention may include, but are not limited to diabetes. It is also contemplated by the present application that changes in the abundance of the identified markers of Table 1 are indicative of type 1 diabetes in a subject. Accordingly, the methods described herein may be further used to diagnose type 1 diabetes in a subject.

Therefore, another aspect of the present invention relates to a method of diagnosing type 1 diabetes or an increased risk of type 1 diabetes in a subject. The method includes obtaining a biological sample from the subject and determining a level of one or more of the polypeptide listed in Table 1.

In another aspect, the present invention identifies a set of proteins indicative of type 1 diabetes using high-throughput proteomics technology. The protein markers indicative of type 1 diabetes are listed in Table 1. The biological samples (e.g., urine) obtained from subjects who were afflicted with type 1 diabetes were analyzed. The protein expression level from these samples were then compared to samples obtained from subjects who were not afflicted by diabetes. It was found that the proteins listed in Table 1 can be used to discriminate between subjects with type 1 diabetes and subjects who are not afflicted with type 1 diabetes.

The biological samples (e.g., urine) obtained from subjects afflicted with diabetes compared to samples obtained from subjects not afflicted with type 1 diabetes exhibit differing levels (i.e., increased levels and decreased levels) of the proteins listed in Table 1. Therefore, the expression profiles of the proteins presented in Table 1, can be used to diagnose type 1 diabetes.

It is further contemplated that changes in the abundance of the identified markers of Table 2 are indicative of a sub-type of type 1 diabetes responsive to treatment by an angiotensin-converting enzyme inhibitor and/or an angiotensin receptor blocker. Accordingly, the methods described herein may be further used to diagnose a subtype of type 1 diabetes responsive to treatment by an angiotensin-converting enzyme inhibitor and/or an angiotensin receptor blocker in a subject.

The present invention provides the identity of a set of proteins indicative of a sub-type of type 1 diabetes responsive to treatment by an angiotensin-converting enzyme inhibitor and/or an angiotensin receptor blocker identified using high-throughput proteomics technology. The protein markers indicative of a sub-type of type 1 diabetes responsive to treatment by an angiotensin-converting enzyme inhibitor and/or an angiotensin receptor blocker are listed in Table 2. By way of example, samples of urine obtained from subjects who were afflicted with type 1 diabetes can be analyzed. The protein expression level from these samples can be compared to samples obtained from subjects who were afflicted by a sub-type of type 1 diabetes responsive to treatment by an angiotensin-converting enzyme inhibitor and/or an angiotensin receptor blocker. The proteins listed in Table 2 can then be used to discriminate between subjects with type 1 diabetes and subjects who are afflicted by a sub-type of type 1 diabetes responsive to treatment by an angiotensin-converting enzyme inhibitor and/or an angiotensin receptor blocker.

The samples of urine obtained from subjects afflicted with diabetes compared to samples of urine obtained from subjects afflicted by a sub-type of type 1 diabetes responsive to treatment by an angiotensin-converting enzyme inhibitor and/or an angiotensin receptor blocker exhibit differing levels (i.e., increased levels and decreased levels) of the proteins listed in Table 2. Therefore, the expression profiles of the proteins presented in Table 2, can be used to diagnose a sub-type of type 1 diabetes responsive to treatment by an angiotensin-converting enzyme inhibitor and/or an angiotensin receptor blocker.

The application further relates to the use of expression profiles of the marker proteins listed in Table 4 in a method for diagnosing an increased risk of development of renal function decline and/or end-stage renal disease ESRD in a subject with type-1 diabetes. The method includes: (1) obtaining a biological sample from a subject; and (2) determining, in the biological sample, the level of one or more of polypeptides selected from the group consisting of the proteins presented in Table 4 (i.e., Isofrom 1 of Serum albumin, Protein AMBP, Apolipoprotein D, alpha-2-glycoprotein 1, zinc, CD59 glycoprotein, Putative uncharacterized protein CD99, cDNA FLJ57622, highly similar to Clusterin, Ceruloplasmin, Isoform 1 of Fibrinogen alpha chain, Basement membrane-specific heparin sulfate proteoglycan core protein, IGHM protein, IGLV4-3 protein, Isoform LMW of Kininogen-1, Keratin, type II cytoskeletal 1, Keratin, type I cytoskeletal 9, Matrix Gla protein, cDNA FLJ59142, highly similar to Epididymal secretory protein E1, Alpha-1-acid glycoprotein1, Prolactin-inducible protein, Prosaposin, Prostaglandin-H2 D-isomerase, Isoform 1 of Semenogelin-1, Semenogelin-2, Isoform 1 of Alpha-1-antitryspin, Antithrombin III variant, Plasma protease C1 inhibitor, Anchor protein, Secreted Ly-6/uPAR-related protein 1, Protein TET1, Serotransferrin, Isoform 1 of Uromodulin, Neurosecretory VGF, precursor, analogs and fragments thereof), wherein an increase or decrease in the level of one or more of the proteins compared to a control level is indicative of the subject with type 1 diabetes having an increased risk of renal function decline and/or ESRD.

A further aspect of the application relates to the use of expression profiles of the marker proteins listed in Table 5 in a method for diagnosing an increased risk of development of microalbuminuria, renal function decline, and/or end-stage renal disease ESRD in a subject. The method includes: (1) obtaining a biological sample from a subject; and (2) determining, in the biological sample, the level of one or more of polypeptides selected from the group consisting of Tamms Horsfall Protein (THP), progranulin, and alpha-1-acid glycoprotein (AGP), wherein an increase or decrease in the level of one or more of the proteins compared to a control level is indicative of the subject having an increased risk of microalbuminuria, renal function decline, and/or ESRD.

Other diabetes biomarkers contemplated by the present invention include nucleic acid molecules including polynucleotide sequences coding for the inventive protein markers described in Table 1, Table 2, Table 4, and Table 5 (or analogs and fragments thereof) and polynucleotides that hybridize with portions of these nucleic acid molecules.

Information on levels of a given set of biomarkers obtained using biological samples from individuals afflicted with diabetes may be grouped to form a diabetes expression profile map. Preferably, a diabetes expression profile map results from the study of a large number of individuals with the same disease sub-type. In certain embodiments, a diabetes expression profile map is established using samples from individuals with matched age, sex, and body index. Each expression profile map provides a template for comparison to biomarker expression patterns generated from unknown biological samples. Diabetes expression profile maps may be presented as a graphical representation (e.g., on paper or a computer screen), a physical representation (e.g., a gel or array) or a digital representation stored in a computer-readable medium.

As will be appreciated by those of ordinary skill in the art, sets of biomarkers whose expression profiles correlate with diabetes or a sub-type of diabetes may be used to identify, study, or characterize unknown biological samples. Accordingly, in one aspect of the present invention, methods for characterizing biological samples obtained from a subject suspected of having diabetes, for diagnosing diabetes in a subject, and for assessing the responsiveness of diabetes in a subject to treatment are contemplated. In such methods, the biomarkers' expression levels determined for a biological sample, obtained from the subject, are compared to the levels in one or more control samples. The control samples may be obtained from a healthy individual (or a group of healthy individuals), and/or from an individual (or group of individuals) afflicted with diabetes. As mentioned above, the control expression levels of the biomarkers of interest are preferably determined from a significant number of individuals, and an average or mean is obtained. In certain aspects of the invention, the levels determined for the biological sample under investigation are compared to at least one expression profile map for diabetes, as described above.

The methods of the invention may be applied to the study of any type of biological samples allowing one or more inventive biomarkers to be assayed. Examples of biological samples include, but are not limited to, urine, blood, blood products (e.g., blood plasma), joint fluid, saliva, and synovial fluid. In a particular aspect of the present invention, the biological sample is urine obtained from the subject.

The biological samples used in the practice of the inventive methods may be fresh or frozen samples collected from a subject, or archival samples with known diagnosis, treatment and/or outcome history. Biological samples may be collected by any non-invasive means, such as, for example, by collecting a subject's urine sample. In some aspects, a urine sample can be a midstream urine sample taken from a subject to avoid possible contamination of the forestream urine. In certain aspects, the inventive methods are performed on the biological sample itself without or with limited processing of the sample.

Preferably, there is enough of the biological sample to accurately and reliably determine the abundance of the set of biomarkers of interest. Multiple biological samples may be taken from the subject in order to obtain a representative sampling from the subject.

In still other embodiments, the inventive methods are performed on a protein extract prepared from the biological sample. Preferably, the protein extract contains the total protein content. However, the methods may also be performed on extracts containing one or more of: membrane proteins, nuclear proteins, and cytosolic proteins. Methods of protein extraction are well known in the art (see, for example “Protein Methods”, D. M. Bollag et al., 2nd Ed., 1996, Wiley-Liss; “Protein Purification Methods: A Practical ApprIPSch”, E. L. Harris and S. Angal (Eds.), 1989; “Protein Purification Techniques: A Practical Approach”, S. Roe, 2nd Ed., 2001, Oxford University Press; “Principles and Reactions o/Protein Extraction, Purification, and Characterization”, H. Ahmed, 2005, CRC Press: Boca Raton, Fla.). Numerous different and versatile kits can be used to extract proteins from bodily fluids and tissues, and are commercially available from, for example, BioRad Laboratories (Hercules, Calif), BD Biosciences Clontech (Mountain View, Calif.), Chemicon International, Inc. (Temecula, Calif), Calbiochem (San Diego, Calif.), Pierce Biotechnology (Rockford, Ill.), and Invitrogen Corp. (Carlsbad, Calif.). User Guides that describe in great detail the protocol to be followed are usually included in all these kits. Sensitivity, processing time and costs may be different from one kit to another. One of ordinary skill in the art can easily select the kits most appropriate for a particular situation. After the protein extract has been obtained, the protein concentration of the extract is preferably standardized to a value being the same as that of the control sample in order to allow signals of the protein markers to be quantitated. Such standardization can be made using photometric or spectrometric methods or gel electrophoresis.

In yet other aspects, the inventive methods are performed on nucleic acid molecules extracted from the biological sample. For example, RNA may be extracted from the sample before analysis. Methods of RNA extraction are well known in the art (see, for example, J. Sambrook et al., “Molecular Cloning: A Laboratory Manual”, 1989, 2nd Ed., Cold Spring Harbor Laboratory Press: Cold Spring Harbor, N.Y.). Most methods of RNA isolation from bodily fluids or tissues are based on the disruption of the tissue in the presence of protein denaturants to quickly and effectively inactivate RNAses. Isolated total RNA may then be further purified from the protein contaminants and concentrated by selective ethanol precipitations, phenol/chloroform extractions followed by isopropanol precipitation or cesium chloride, lithium chloride or cesium trifluoroacetate gradient centrifugations. Kits are also available to extract RNA (i.e., total RNA or mRNA) from bodily fluids or tissues and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), GIBCO BRL (Gaithersburg, Md.), and Qiagen, Inc. (Valencia, Calif).

In certain aspects, after extraction, mRNA is amplified, and transcribed into cDNA, which can then serve as template for multiple rounds of transcription by the appropriate RNA polymerase. Amplification methods are well known in the art (see, for example, A. R. Kimmel and S. L. Berger, Methods Enzymol. 1987, 152: 307-316; J. Sambrook et al., “Molecular Cloning: A Laboratory Manual”, 1989, 2nd Ed., Cold Spring Harbour Laboratory Press: New York; “Short Protocols in Molecular Biology”, F. M. Ausubel (Ed.), 2002, 5th Ed., John Wiley & Sons; U.S. Pat. Nos. 4,683,195; 4,683,202 and 4,800,159). Reverse transcription reactions may be carried out using non-specific primers, such as an anchored oligo-dT primer, or random sequence primers, or using a target-specific primer complementary to the RNA for each probe being monitored, or using thermostable DNApolymerases (such as avian myeloblastosis virus reverse transcriptase or Moloney murine leukemia virus reverse transcriptase).

The diagnostic methods of the present invention generally involve the determination of the abundance levels of a plurality (i.e., one or more, e.g., at least 2, at least 3, at least 4, at least 5, at least 6, at least 7, at least 8, at least 9, at least 10 or more) of polypeptides in a biological sample obtained from a subject. Determination of protein levels in the practice of the inventive methods may be performed by any suitable method (see, for example, E. Harlow and A. Lane, “Antibodies: A Laboratories Manual”, 1988, Cold Spring Harbor Laboratory: Cold Spring Harbor, N.Y.).

In general, protein levels are determined by contacting a biological sample isolated from a subject with binding agents for one or more of the protein markers listed in Table 1, Table 2; Table 4, and/or Table 5 determining, in the sample, the levels of polypeptides that bind to the binding agents; and comparing the levels of polypeptides in the sample with the levels of polypeptides in a control sample. As used herein, the term “binding agent” refers to an entity such as a polypeptide or antibody that specifically binds to an inventive protein marker. An entity “specifically binds” to a polypeptide if it reacts/interacts at a detectable level with the polypeptide but does not react/interact detectably with peptides containing unrelated sequences or sequences of different polypeptides.

In certain aspects of the invention, the binding agent is a ribosome, with or without a peptide component, an RNA molecule, or a polypeptide (e.g., a polypeptide that comprises a polypeptide sequence of a protein marker, a peptide variant thereof, or a non-peptide mimetic of such a sequence).

In other aspects, the binding agent is an antibody specific for a protein marker of the invention. Suitable antibodies for use in the methods of the present invention include monoclonal and polyclonal antibodies, immunologically active fragments (e.g., Fab or (Fab)2 fragments), antibody heavy chains, humanized antibodies, antibody light chains, and chimeric antibodies. Antibodies, including monoclonal and polyclonal antibodies, fragments and chimeras, may be prepared using methods known in the art (see, for example, R. G. Mage and E. Lamoyi, in “Monoclonal Antibody Production Techniques and Applications”, 1987, Marcel Dekker, Inc.: New York, pp. 79-97; G. Kohler and C. Milstein, Nature, 1975, 256: 495-497; D. Kozbor et al., J. Immunol. Methods, 1985,81: 31-42; and R. J. Cote et al., Proc. Natl. Acad. Sci. 1983, 80: 2026-203; R. A. Lerner, Nature, 1982, 299: 593-596; A. C. Nairn et al., Nature, 1982,299: 734-736; A. J. Czernik et al., Methods Enzymol. 1991, 201: 264-283; A. J. Czernik et al., Neuromethods: Regulatory Protein Modification: Techniques & Protocols, 1997, 30: 219-250; A. J. Czemik et al., NeuroNeuroprotocols, 1995, 6: 56-61; H. Zhang et al., J. Biol. Chern. 2002, 277: 39379-39387; S. L. Morrison et al., Proc. Natl. Acad. Sci., 1984, 81: 6851-6855; M. S. Neuberger et al., Nature, 1984,312: 604-608; S. Takeda et al., Nature, 1985, 314: 452-454). Antibodies to be used in the methods of the invention can be purified by methods well known in the art (see, for example, S. A. Minden, “Monoclonal Antibody Purification”, 1996, IBC Biomedical Library Series: Southbridge, Mass.). For example, antibodies can be affinity purified by passage over a column to which a protein marker or fragment thereof is bound. The bound antibodies can then be eluted from the column using a buffer with a high salt concentration.

Instead of being prepared, antibodies to be used in the methods of the present invention may be obtained from scientific or commercial sources.

In certain embodiments, the binding agent is directly or indirectly labeled with a detectable moiety. The role of a detectable agent is to facilitate the detection step of the diagnostic method by allowing visualization of the complex formed by binding of the binding agent to the protein marker (or analog or fragment thereof). Preferably, the detectable agent is selected such that it generates a signal which can be measured and whose intensity is related (preferably proportional) to the amount of protein marker present in the sample being analyzed. Methods for labeling biological molecules such as polypeptides and antibodies are well-known in the art (see, for example, “Affinity Techniques. Enzyme Purification. Part B”, Methods in Enzymol., 1974, Vol. 34, W. B. Jakoby and M. Wilneck (Eds.), Academic Press: New York, N.Y.; and M. Wilchek and E. A. Bayer, Anal. Biochem., 1988, 171: 1-32).

Any of a wide variety of detectable agents can be used in the practice of the present invention. Suitable detectable agents include, but are not limited to: various ligands, radionuclides, fluorescent dyes, chemiluminescent agents, microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available.

In certain aspects, the binding agents (e.g., antibodies) may be immobilized on a carrier or support (e.g., a bead, a magnetic particle, a latex particle, a microtiter plate well, a cuvette, or other reaction vessel). Examples of suitable carrier or support materials include agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinylether-maleic acid copolymer, amino acid copolymer, ethylene-maleic acid copolymer, nylon, silk, and the like. Binding agents may be indirectly immobilized using second binding agents specific for the first binding agents (e.g., mouse antibodies specific for the protein markers may be immobilized using sheep anti-mouse IgG Fc fragment specific antibody coated on the carrier or support).

Protein levels in the diagnostic methods of the present invention may be determined using immunoassays. Examples of such assays are radioimmunoassays, enzyme immunoassays (e.g., ELISA), immunofluorescence immunoprecipitation, latex agglutination, hemagglutination, and histochemical tests, which are conventional methods well-known in the art. As will be appreciated by one skilled in the art, the immunoassay may be competitive or noncompetitive. Methods of detection and quantification of the signal generated by the complex formed by binding of the binding agent with the protein marker will depend on the nature of the assay and of the detectable moiety (e.g., fluorescent moiety).

Alternatively, the protein levels may be determined using mass spectrometry based methods or image (including use of labeled ligand) based methods known in the art for the detection of proteins. Other suitable methods include proteomics-based methods. Proteomics, which studies the global changes of protein expression in a sample, typically includes the following steps: (1) separation of individual proteins in a sample by electrophoresis (I-D PAGE), (2) identification of individual proteins recovered from the gel (e.g., by mass spectrometry or N-terminal sequencing), and (3) analysis of the data using bioinformatics.

As already mentioned above, the diagnostic methods of the present invention may involve determination of the expression levels of a set of nucleic acid molecules comprising polynucleotide sequences coding for an inventive protein marker. Determination of expression levels of nucleic acid molecules in the practice of the inventive methods may be performed by any suitable method, including, but not limited to, Southern analysis, Northern analysis, polymerase chain reaction (PCR) (see, for example, U.S. Pat. Nos. 4,683,195; 4,683,202, and 6,040,166; “PCR Protocols: A Guide to Methods and Applications”, Innis et al. (Eds.), 1990, Academic Press: New York), reverse transcriptase PCR(RT-PCT), anchored PCR, competitive PCR (see, for example, U.S. Pat. No. 5,747,251), rapid amplification of cDNA ends (RACE) (see, for example, “Gene Cloning and Analysis: Current Innovations, 1997, pp. 99-115); ligase chain reaction (LCR) (see, for example, EP 01 320308), one-sided PCR (Ohara et al., Proc. Natl. Acad. Sci., 1989, 86: 5673-5677), in situ hybridization, Taqman based assays (Holland et al., Proc. Natl. Acad. Sci., 1991, 88:7276-7280), differential display (see, for example, Liang et al., Nucl. Acid. Res., 1993, 21: 3269-3275) and other RNA fingerprinting techniques, nucleic acid sequence based amplification (NASBA) and other transcription based amplification systems (see, for example, U.S. Pat. Nos. 5,409,818 and 5,554,527), Qbeta Replicase, Strand Displacement Amplification (SDA), Repair Chain Reaction (RCR), nuclease protection assays, subtraction-based methods, Rapid-Scan™, and the like.

Nucleic acid probes for use in the detection of polynucleotide sequences in biological samples may be constructed using conventional methods known in the art. Suitable probes may be based on nucleic acid sequences encoding at least 5 sequential amino acids from regions of nucleic acids encoding a protein marker, and preferably comprise about 15 to about 50 nucleotides. A nucleic acid probe may be labeled with a detectable moiety, as mentioned above in the case of binding agents. The association between the nucleic acid probe and detectable moiety can be covalent or non-covalent. Detectable moieties can be attached directly to nucleic acid probes or indirectly through a linker (E. S. Mansfield et al., Mol. Cell. Probes, 1995, 9: 145-156). Methods for labeling nucleic acid molecules are well-known in the art (for a review of labeling protocols, label detection techniques and recent developments in the field, see, for example, L. J. Kricka, Ann Clin. Biochem. 2002, 39: 114-129; R. P. van Gijlswijk et al., Expert Rev. Mol. Diagn. 2001, 1: 81-91; and S. Joos et al., J. Biotechnol. 1994, 35:135-153).

Nucleic acid probes may be used in hybridization techniques to detect polynucleotides encoding the protein markers. The technique generally involves contacting an incubating nucleic acid molecules in a biological sample obtained from a subject with the nucleic acid probes under conditions such that specific hybridization takes place between the nucleic acid probes and the complementary sequences in the nucleic acid molecules. After incubation, the non-hybridized nucleic acids are removed, and the presence and amount of nucleic acids that have hybridized to the probes are detected and quantified.

Detection of nucleic acid molecules comprising polynucleotide sequences coding for a protein marker may involve amplification of specific polynucleotide sequences using an amplification method such as PCR, followed by analysis of the amplified molecules using techniques known in the art. Suitable primers can be routinely designed by one skilled in the art. In order to maximize hybridization under assay conditions, primers and probes employed in the methods of the invention generally have at least 60%, preferably at least 75% and more preferably at least 90% identity to a portion of nucleic acids encoding a protein marker.

Hybridization and amplification techniques described herein may be used to assay qualitative and quantitative aspects of expression of nucleic acid molecules comprising polynucleotide sequences coding for the inventive protein markers.

Alternatively, oligonucleotides or longer fragments derived from nucleic acids encoding each protein marker may be used as targets in a microarray. A number of different array configurations and methods of their production are known to those skilled in the art (see, for example, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637). Microarray technology allows for the measurement of the steady-state level of large numbers of polynucleotide sequences simultaneously. Microarrays currently in wide use include cDNA arrays and oligonucleotide arrays. Analyses using microarrays are generally based on measurements of the intensity of the signal received from a labeled probe used to detect a cDNA sequence from the sample that hybridizes to a nucleic acid probe immobilized at a known location on the microarray (see, for example, U.S. Pat. Nos. 6,004,755; 6,218,114; 6,218,122; and 6,271,002). Array-based gene expression methods are known in the art and have been described in numerous scientific publications as well as in patents (see, for example, M. Schena et al., Science, 1995, 270: 467-470; M. Schena et al., Proc. Natl. Acad. Sci. USA 1996, 93: 10614-10619; Chen et al., Genomics, 1998, 51: 313324; U.S. Pat. Nos. 5,143,854; 5,445,934; 5,807,522; 5,837,832; 6,040,138; 6,045,996; 6,284,460; and 6,607,885).

Once the levels of the biomarkers of interest have been determined for the biological sample being analyzed, they are compared to the levels in one or more control samples or to at least one expression profile map for diabetes described herein. Comparison of levels according to methods of the present invention is preferably performed after the levels obtained have been corrected for both differences in the amount of sample assayed and variability in the quality of the sample used (e.g., amount of protein extracted, or amount and quality of mRNA tested). Correction may be carried out using different methods well-known in the art. For example, the protein concentration of a sample may be standardized using photometric or spectrometric methods or gel electrophoresis (as already mentioned above) before the sample is analyzed. In case of samples containing nucleic acid molecules, correction may be carried out by normalizing the levels against reference genes (e.g., housekeeping genes) in the same sample. Alternatively or additionally, normalization can be based on the mean or median signal (e.g., Ct in the case of RT-PCR) of all assayed genes or a large subset thereof (global normalization approach).

For a given set of biomarkers, comparison of an expression pattern obtained for a biological sample against an expression profile map established for diabetes or a particular subtype of diabetes may comprise comparison of the normalized levels on a biomarker-by-biomarker basis and/or comparison of ratios of levels within the set of biomarkers. In addition, the protein expression pattern obtained for the biological sample being analyzed, may be compared against each of the expression profile maps (e.g., expression profile map for non-diabetes, expression profile map for diabetes, expression profile map for type 1 diabetes, expression profile map for a sub-type of type 1 diabetes responsive to treatment by an angiotensin-converting enzyme inhibitor and/or an angiotensin receptor blocker or against an expression profile that defines delineations made based upon all the diabetes expression profile maps.

Using methods described herein, skilled physicians may select and prescribe treatments adapted to each individual subject based on the diagnosis of a renal disorder, diabetes, and/or type 1 diabetes provided to the subject through determination of the levels of the inventive biomarkers. In particular, the present invention provides physicians with a non-subjective means to diagnose type 1 diabetes, which will allow for early treatment, when intervention is likely to have its greatest effect. Selection of an appropriate therapeutic regimen for a given patient may be made based solely on the diagnosis provided by the inventive methods. Alternatively, the physician may also consider other clinical or pathological parameters used in existing methods to diagnose diabetes and assess its advancement.

In some aspects, diabetes diagnosed by the methods of the present invention, may be a sub-type of type 1 diabetes treated with an angiotensin receptor blocker (ARB) and/or angiotensin-converting enzyme inhibitor (ACE-I). Studies have shown that the ACE-I/ARB treatment in type 1 diabetes patients with albuminuria is associated with lower odds of progression of coronary artery calcification (Maahs et al., ACE-I/ARB treatment in type 1 diabetes patients with albuminuria is associated with lower odds of progression of coronary artery calcification. J. Diabetes Complications 21(5):273-279, 2007). In addition, Laffel et al. have reported the beneficial effect of ACE-I with captopril on diabetic nephropathy in normotensive type 1 diabetes patients with microalbuminuria (Laffel et al. Am J Med 99(5):497-504).

It is further contemplated by the present invention that once a subject is diagnosed using the methods described above, an ACE-I and/or ARB may be administered to treat the subject having diabetes. In one particular example, a subject can be administered an ACE-I and/or an ARB when protein levels in a biological sample obtained from the subject, define a sub-type of diabetes that is responsive to treatment with an ACE-I and/or ARB.

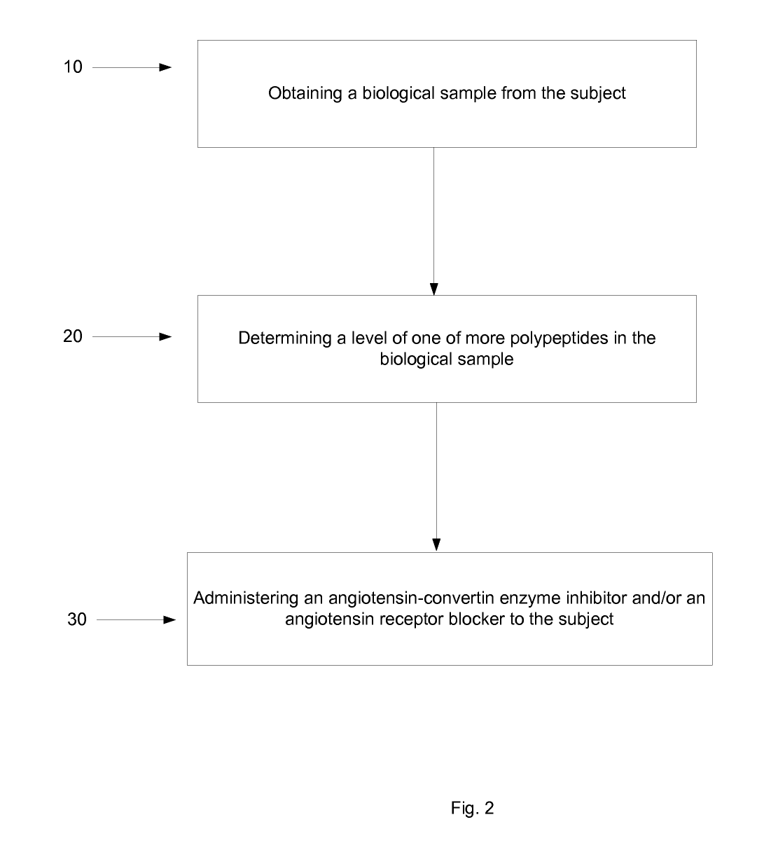

Therefore, as shown in FIG. 2 at 30, in some aspects of the invention an ACE-I and/or ARB, may be used to treat diabetes in the subject who has been diagnosed with diabetes that is responsive to treatment by an ACE-I and/or ARB using the method described herein. The ACE-I administered to the subject can include, but is not limited to, Benazepril, Captopril, Enalapril, Fosinopril, Lisinopril, Moexipril, Perindopril, Quinapril, Ramipril, and Trandolapril. The ARB administered to the subject can include, but is not limited to Losartan, Telmisartan, Irbesartan, Olmesartan, and Valsartan, Candesartan, and Eprosartan.

Effective dosages and administration regimens can be readily determined by good medical practice and the clinical condition of the individual subject. The frequency of administration will depend on the pharmacokinetic parameters of the active ingredient(s) and the route of administration. The optimal pharmaceutical formulation can be determined depending upon the route of administration and desired dosage. Such formulations may influence the physical state, stability, rate of in vivo release, and rate of in vivo clearance of the administered compounds.

Depending on the route of administration, a suitable dose may be calculated according to body weight, body surface area, or organ size. Optimization of the appropriate dosage can readily be made by those skilled in the art in light of pharmacokinetic data observed in human clinical trials. The final dosage regimen will be determined by the attending physician, considering various factors which modify the action of drugs, e.g., the drug's specific activity, the severity of the damage and the responsiveness of the patient, the age, condition, body weight, sex and diet of the patient, the severity of any present infection, time of administration and other clinical factors.

In another aspect, the present invention provides kits comprising materials useful for carrying out diagnostic methods according to the present invention. The diagnosis and sub-typing procedures described herein may be performed by diagnostic laboratories, experimental laboratories, or practitioners. The invention provides kits, which can be used in these different settings.

Materials and reagents for characterizing biological samples, diagnosing diabetes, and/or sub-typing diabetes in a subject according to the inventive methods may be assembled together in a kit. In certain aspects, an inventive kit comprises at least one reagent that specifically detects levels of one or more inventive biomarkers, and instructions for using the kit according to a method of the invention. Each kit may preferably include the reagent, which renders the procedure specific. Thus, for detecting/quantifying a protein marker (or an analog or fragment thereof), the reagent that specifically detects levels of the protein may be an antibody that specifically binds to the protein marker (or analog or fragment thereof). For detecting/quantifying a nucleic acid molecule comprising a polynucleotide sequence coding a protein marker, the reagent that specifically detects expression levels may be a nucleic acid probe complementary to the polynucleotide sequence (e.g., cDNA or an oligonucleotide). The nucleic acid probe may or may not be immobilized on a substrate surface (e.g., beads, a microarray, and the like).

Depending on the procedure, the kit may further comprise one or more of, extraction buffer and/or reagents, amplification buffer and/or reagents, hybridization buffer and/or reagents, immunodetection buffer and/or reagents, labeling buffer and/or reagents, and detection means. Protocols for using these buffers and reagents for performing different steps of the procedure may be included in the kit.

The reagents may be supplied in a solid (e.g., lyophilized) or liquid form. The kits of the present invention may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the disclosed methods may also be provided. The individual containers of the kit are preferably maintained in close confinement for commercial sale.

In certain aspects, the kits of the present invention further include control samples. In other aspects of the invention, the inventive kits include at least one expression profile map for diabetes and/or diabetes sub-type as described herein for use as comparison template. Preferably, the expression profile map is digital information stored in a computer-readable medium.

Instructions for using the kit, according to one or more methods of the invention, may comprise instructions for processing the biological sample obtained from the subject, and/or for performing the test, instructions for interpreting the results. As well as a notice in the form prescribed by a governmental agency (e.g., FDA) regulating the manufacture, use or sale of pharmaceuticals or biological products.

### EXAMPLE 1

### Biomarkers Indicative of Diabetes

Understanding changes in protein abundance, modifications, and iso-forms not only informs the pathophysiological basis of disease and health, the reliable and reproducible detection of such changes in proteins can be utilized as biomarkers for the presence of disease and its remission. Although no single technique can mine all the complexity of the proteome derived from the range of available biological samples in a single type of experiment, a number of standard approaches have evolved that are extremely valuable and reliable in quantifying protein abundance for biomarker discovery. One such method is label-free protein expression which capitalized on the recent advances in chromatographic reproducibility and high-resolution mass spectrometry instrumentation now available which have resulted in a blossoming of quantification methods for proteomics studies. This method includes clustering of identified peptides across multiple samples as well as accurate mass tag database approaches to identification, in both cases coupled to LC-MS based quantification. These approaches provide very valuable weapons in the arsenal of proteomics techniques for biomarker discovery as they have good proteome coverage and permit an array of statistical approaches suited to the examination of high dimensional data. While this technique is a powerful approach for discovery based studies, it is not suitable for verification/validation studies where larger sample numbers are required. Therefore, protein targets selected from label free expression studies are transferred to validation assays, which have higher throughput. Two types of validation assays employed are the enzyme-linked immunosorbent assay (ELISA) which employs antibodies to the protein target for detection or a multiple reaction monitoring (MRM) assay, which is a mass spectrometry detection technique that quantifies peptides from the protein of interest. Both assays use calibrant standards to generate concentration curves from which absolute protein quantification from a sample can be derived. These techniques have thus been employed in the discovery of urine biomarkers listed below.

### Methods and Materials

A label free expression experiment was performed to identify proteins that discriminate type 1 diabetes from healthy controls in a human cohort. Using this data, we have defined a panel of protein biomarkers, which can serve as indicators of diabetes. Four milliliters from nine human urine samples (3 diabetic, 3 control, and 3 macro albuminuric) were prepared and were analyzed by label free protein expression. Using a small amount of the total digest (100 nanograms) we tracked and quantified over 950 peptides. Statistical analysis highlighted over 31 different proteins shown in Table 1, which discriminate diabetic patients from healthy controls. These represent proteins whose abundance in urine change due to disease and for which antibody based assays such as ELISA can be developed for clinical detection. Our data shows that there are reliable and consistent urinary protein and peptide profiles in diabetic patients. These are derived from both changes in overall abundance in proteins as well as differences in processing of proteins, which is reflected at the peptide level.

TABLE 1

| | | Direction of |
|---|---|---|
| Protein.ID | Protein Description | Change |
| IPI00013945 | UMOD Isoform 1 of Uromodulin | Down |
| IPI00022463 | TF Serotransferrin precursor | Down |
| IPI00025273 | GART Isoform Long of Trifunctional purine | Down |
| | biosynthetic protein adenosine-3 | |
| IPI00032325 | CSTA Cystatin-A | Down |
| IPI00166729 | AZGP1 alpha-2-glycoprotein, 1 zinc | Down |
| IPI00179330 | RPS27A; UBC; UBB ubiquitin and ribosomal protein | Down |
| | S27a precursor | |
| IPI00184855 | C16orf73 Novel protein | Down |
| IPI00221090 | ZFHX2 Zinc finger homeobox protein 2 | Down |
| IPI00298980 | CHRM1Muscarinic acetylcholine receptor M1 | Down |
| IPI00329787 | WDR20WD repeat domain 20 isoform 3 | Down |
| IPI00382781 | IL20RA Interleukin 20 receptor, alpha | Down |
| IPI00644454 | SORCS1 Isoform 4 of VPS10 domain-containing | Down |
| | receptor SorCS1 precursor | |
| IPI00748008 | CDNA FLI41726 fis, clone HLUNG2014449 | Down |
| IPI00783623 | Similar to CF3558-PA, isoform A | Down |
| IPI00000655 | SLC16A2 Solute carrier family 16, member 2 | Up |
| IPI00018909 | TFF3 trefoil factor 3 precursor | Up |
| IPI00019449 | RNASE2 Nonsecretory ribonuclease precursor | Up |
| IPI00022620 | SLURP1 Secreted Ly-6/upar-related protein 1 precursor | Up |
| IPI00104396 | RGSL1 Hypothetical Protein | Up |
| IPI00215894 | KNG1 Isoform LMW of Kininogen-1 precursor | Up |
| IPI00218705 | KIR2DL4 Isoform 3 of Killer cell immunoglobulin-like | Up |
| | receptor 2DL4 precursor | |
| IPI00297989 | MAGEB6 Melanoma-associated antigen B6 | Up |
| IPI00304577 | AP2A1 isoform A of AP-2 complex subunit alpha-1 | Up |
| IPI00306339 | SPP1 secreted phosphoprotein 1 isoform b | Up |
| IPI00307483 | CYP4F11 Cytochrome P450 4F11 | Up |
| IPI00457301 | IGFL4 Insulin growth factor-like family member 4 | Up |
| | precursor | |
| IPI00478086 | SLC39A7 Non-class II protein | Up |
| IPI00550720 | C19orf57 Uncharacterized protein C19orf 57 | Up |
| IPI00553177 | SERPINA Alpha-1-antitrypsin precursor | Up |

### EXAMPLE 2

### Biomarkers Indicative of Kidney Dysfunction

This Example outlines proteomic methods to examine the abundance of key proteins in human urine to be used as a routine screening test for detecting increased risk of complications in Type 1 and 2 DM patients as well as patients with hypertension. These urinary markers reflect early changes in GFR and biological changes in kidney that indicates progression of renal disease and risk for CAD. The indicated markers can be used as prognostic indicators to trigger standard and effective therapeutic interventions with angiotensin converting enzyme inhibitor (ACE-I) and/or angiotensin receptor blocker (ARB) treatments earlier than currently prescribed, likely lowering the risk of both ESRD and CAD and providing significant costs savings to health care systems and improving patient outcomes. This Example outlines the specific markers and assays in human urine samples to detect these markers by mass spectrometry, ELISA, or related antibody and other methods.

### Methods and Materials

The procedures specific to the human study disclosed here are summarized as follows. Nine human urine samples (3 type 1 diabetic, 3 control, and 3 type 1 diabetic having progressed to micro- or macro-albuminuric) from the Coronary Artery Calcification in Type 1 Diabetes (CACTI) study were desalted using a microcon concentrator, the protein concentration measured and proteolytic digestion was performed with Lys-C (Wako Chemicals, Richmond Va.) for 18 hours at 37° C. This removal of salt makes the sample easily analyzed by mass spectrometry while the concentration and adjustment of protein values across samples provides a standardization that makes comparison of different urine samples easier, as urine collected from different patients at different times may have quite varied concentrations. Sixty nanograms of each sample were analyzed by LC/MS and the order of sample injections randomized Separation of peptides via capillary liquid chromatography was performed using a Dionex Ultimate 3000 capillary LC system (Dionex Sunnyvale, Calif.). Mass spectrometry analyses of samples were performed using a Fourier Transform LTQ (Thermo Waltham, Mass.). Positive mode electrospray was conducted using a nebulized nanoflow sprayer and the mass spectrometer was operated at a resolution of 25 kDa. Quantitative and qualitative data were acquired using alternating full MS scan and MS/MS scans. Survey data were acquired from m/z of 400 to 1600 and up to 3 precursors were interrogated by MS/MS per switch. The switch into MS/MS was based on precursor intensity and 2 scans were acquired for every precursor interrogated. Nine samples were analyzed by LC/MS/MS. Excellent chromatographic reproducibility was observed across injections with retention times on average deviating on the order of +/−1 minute. Raw LC/MS/MS data was processed via Proteomarker software (Inforchromics, Toronto, Canada) to provide peak lists that were subsequently searched by Mascot version 2.2.0 (Matrix Science London, UK). The database used was a compilation of human and mouse databases from the International Protein Index. The criteria for peptide identification were a mass accuracy of ≦10 ppm and an expectation value of P≦0.05. Proteins that had >2 peptides matching the above criteria were considered confirmed assignments. Automated differential quantification of peptides in a set of samples was accomplished by ProteoMarker. This software suite automates quantitative comparison of MS data sets. Peptides that were significant were identified using statistical methods.

### Results

Using a small amount of the total digest (100 nanograms) we tracked and quantified over 950 peptides and compared their abundance among the samples. Statistical analysis has highlighted over 374 peptides that discriminate T1DM patients without microalbuminuria from T1DM patients with microalbuminuria. Thus, as patients progress from diabetic to a condition where risk of complications is confirmed (microalbuminuria) and treatment would be indicated (increased glycemic control and/or ACE-I/ARB inhibitors), the levels of these 374 peptides in a digested urine sample are expected to change significantly (either up or down as indicated) and are surrogate markers for treatment indication.

Two hundred and fifty four of these peptides are derived from proteins from which more than one peptide for that protein was observed in our analysis. Also, the change in abundance (up or down) was consistent for these multiple peptides, thus these 44 proteins and their increase or decrease as a function of complications risk are considered confirmed. Table 2 highlights the 31 proteins indicated as being decreased in urine and the 13 proteins indicated as being increased as a function of disease progression. These represent proteins for which antibody based assays (using the specific proteins or any peptide or protein fragment derived from these sequences as the source as antigens) can be developed for clinical detection. One of the up-regulated proteins is albumin, the current gold standard for diagnosis using a urine assay. However, the changes in ratios of these markers, for example, albumin (numerator) versus uromodulin (UMOD I, denominator) are suggested as an improved predictor of risk. In general, ratios of values of protein levels significantly increased vs. values of protein levels significantly decreased in Table 2, or their reciprocals are likely to represent more sensitive biomarkers.

In addition to the 44 proteins indicated as potential diagnostics, the 374 individual peptides identified by the label free analysis are also sensitive indicators of renal and CAD complications; 254 of these peptides are derived from the 44 proteins in Table 2, and assays based on antibodies developed specifically against these 254 peptide epitopes or assays based on mass spectrometric detection of the 254 peptides in urine whose proteins have been digested by a specific protease would provide useful diagnostic tests.

TABLE 2

| | | Direction of |
|---|---|---|
| Protein I.D. | Protein Description | Change |
| IPI00298828 | APOH Beta-2-glycoprotein 1 precursor | Down |
| IPI00011302 | CD59 glycoprotein 1 precursor | Down |
| IPI00220117 | CD99 Isoform II of CD99 antigen precursor | Down |
| IPI00185662 | CD99L2 protein DKFZp761H2024 | Down |
| IPI00795633 | CLU | Down |
| IPI00297646 | COL1A1 Collagen alpha-1(1) chain precursor | Down |
| IPI00032325 | CSTA Cystatin-A | Down |
| IPI00022290 | DEFB1 Beta-defensin 1 precursor | Down |
| IPI00182138 | GRN Isoform 2 of Granulins precursor | Down |
| IPI00024284 | HSPG2 Basement membrane-specific heparin sulfate | Down |
| | proteoglycan core protein precursor | |
| IPI00845354 | IGKC protein | Down |
| IPI00305461 | ITIH2 Inter-alpha-trypsin inhibitor heavy chain H2 | Down |
| | precursor | |
| IPI00215894 | KNG1 Isoform LMW of Kininogen-1 precursor | Down |
| IPI00301143 | PI16 Isoform 1 of Peptidase inhibitor 16 precursor | Down |
| IPI00004573 | PIGR Polymeric-immunoglobulin receptor precursor | Down |
| IPI00829813 | PIK3IP1 Isoform 2 of Phosphoinositide-3-kinase- | Down |
| | interacting protein 1 precursor | |
| IPI00012503 | PSAP isoform Sap-mu-0 of Proactivator polypeptide | Down |
| | precursor | |
| IPI00013179 | PTGDS Prostaglandin-H2 D-isomerase precursor | Down |
| IPI00023591 | PURA Transcriptional activator protein Pur-Alpha | Down |
| IPI00014048 | RNASE1 Ribonuclease pancreatic precursor | Down |
| IPI00019449 | RNASE2 Nonsecretory ribonuclease precursor | Down |
| IPI00179330 | RPS27A; UBC; UBB ubiquitin and ribosomal protein S27a | Down |
| | precursor | |
| IPI00022620 | SLURP1 Secreted Ly-6/uPar-related protein 1 precursor | Down |
| IPI00306339 | SPP1 secreted phosphoprotein 1 isoform b | Down |
| IPI00010675 | TFF2 Trefoil factor 2 precursor | Down |
| IPI00013945 | UMOD Isoform 1 of Uromodulin precursor | Down |
| IPI00069058 | VGF nerve growth factor inducible precursor | Down |
| IPI00291488 | WFDC2 Isofrom 1 of WAP four-disulfide core domain | Down |
| | protein 2 precursor | |
| IPI00745872 | ALB Isoform 1 of Serum albumin precursor | Up |
| IPI00022426 | AMBP protein precursor | Up |
| IPI00218918 | ANXA1 Annexin A1 | Up |
| IPI00166729 | AZGP1 alpha-2-glycoprotein 1, zinc | Up |
| IPI00004656 | B2M Beta-2-microglobulin precursor | Up |
| IPI00827754 | C gamma 3 | Up |
| IPI00017601 | CP Ceruloplasmin precursor | Up |
| IPI00022488 | HPX Hemopexin precursor | Up |
| IPI00747373 | MUC5B Mucin-5B precursor | Up |
| IPI00020091 | ORM2 Alpha-1-acid glycoprotein 2 precursor | Up |
| IPI00553177 | SERPINA1 Alpha-1-antitrypsin precursor | Up |
| IPI00022463 | TF Serotransferrin precursor | Up |

The 374 specific peptides we detected that had significant increases or decreases in abundance. One hundred twenty of these peptides were the only peptide detected for a protein of interest. These peptides may only be relevant for diagnosis in a peptide-based assay of digested urinary proteins or in an antibody based assay directed against the specific peptides, as their abundance may be driven by protein processing in the uro-genital system. Clinical determination of these peptide biomarkers would employ both antibody and/or mass spectrometry based techniques as indicated above.

### EXAMPLE 3

In this Example, we have performed a longitudinal label free protein expression study to discover early biomarkers of ESRD prior to the onset of disease. Label free protein expression is a peptide based proteomic technique which capitalizes on the highly reproducible chromatography and accurate mass accuracy available in current liquid chromatography/mass spectrometry (LC/MS) systems. This platform quantifies a peptide by its intensity and groups each peptide across individual samples based on its accurate mass and retention time. These intensities are organized into peptide array tables that may be further processed using statistical techniques. Here we describe a label free protein expression analysis in adults with type 1 diabetes who developed microalbuminuria (MA) or a significant decline in estimated GFR over six years of follow-up. We determined a panel of proteins which can serve as novel biomarkers of early development of renal disease. In addition, we verified a selected panel of proteins identified from the discovery analysis via enzyme-linked immunosorbent assays (ELISA) in a larger cohort of T1DM with MA and/or early renal decline.

### Research Design and Methods

### Study Participants

The Coronary Artery Calcification in Type 1 Diabetes (CACTI) study enrolled 652 T1D patients meeting the following criteria: Age 19-56, no history of myocardial infarction, angioplasty, CABG, or angina, currently on insulin therapy, diagnosed before age 30 or a clinical course consistent with type 1 diabetes, on insulin therapy within the first year after diagnosis, and longstanding type 1 diabetes (range of duration 4-52 years). This cohort represents nearly 40% of eligible patients in the Denver Metro Area. Study participants were examined at baseline, 3 years, and 6 years. There were 465 study participants with T1D and 714 study participants without diabetes who were normoalbuminuric at baseline. Over 6 years of follow-up, 25 study participants with T1D who were initially normoalbuminuric developed micro- or macroalbuminuria, and we randomly selected 15 of these study participants to conduct a proteomic discovery phase. Participants with type 1 diabetes who remained normoalbuminuric were frequency matched on age and diabetes duration (N=15).

### Urine Samples

### Experimental Designs

Patients included in the label free discovery study were normalbuminuric at the initial visit (n=29). The Non-progressors were defined as TIDM who did not progress to MA over the course of the study (n=15) and the Progressors were TIDM MA patients who progressed to MA at visit 2 and or visit 3 (n=14). The second study was the ELISA verification study. This study analyzed urine from 110 patients at the baseline visit (visit 1). It included 35 healthy controls and 75 TIDM. The 75 TIDM included: 48 TIDM who did not develop MA over the course of the study, 19 TIDM who did develop MA at visit 2 and/or 3, and 13 TIDM who had early renal function decline (ERFD). Supplemental data table 1 and 2 highlights the experimental units for the discovery and verification studies.

### Label Free Expression

### Sample Preparation

Four milliliters of urine from the Coronary Artery Calcification in Type 1 Diabetes (CACTI) study were desalted using a Microcon concentrator. Each sample is buffer exchanged three times using 3 mL of 50 mM Tris pH 8.8 to a final volume of approximately 100 microliters and protein concentrations are determined by 2D Quant Kit (GE Healthcare Piscataway, N.J.). Ten micrograms of each sample were loaded onto a one dimensional SDS-PAGE gel (4-20% Tris-HCL) as a quality control measure for the desalting step. Subsequent to digestion, each sample was adjusted to 10 micrograms in 50 microliters. Ten microliters of 0.2% Rapigest (Waters, Milford, Mass.) and dithiotheritol to a final concentration of 5 mM was added. The samples were reduced at 80° C. for 15 minutes and cooled to room temperature prior to alkylation with iodacetamide at a final concentration of 10 mM for 30 minutes. Proteolytic digestion was performed with endopeptidase Lys C (Wako Chemicals, Richmond, Va.) with a final enzyme to protein ratio of 1:10 (w/w) for 18 hours at 37° C.

### Liquid Chromatography and Mass Spectrometry

One hundred nanograms of each sample were analyzed by LC/MS/MS and the order of sample injections randomized over all samples. Separation and detection of peptides was performed. Raw LC/MS/MS data was processed via Proteomarker software (Infochromics, Toronto, Canada).

### Data Processing-Qualitative and Quantitative

The raw data for each run were first extracted to provide MS/MS peak lists for identification and intensity based profile peak lists for quantification. The MS/MS peak lists were subsequently searched by Mascot version 2.2.0 (Matrix Science London, UK). The database used was the human International Protein Index (IPI) (68020 sequences). Search settings were as follows: no enzyme specificity, mass accuracy window for precursor ion, 10 ppm; mass accuracy window for fragment ions, 0.8 Daltons; variable modification, including only carbamidomethylation of cysteines and oxidation of methionine. The criteria for peptide identification were a mass accuracy of ≦10 ppm and an expectation value of P≦0.05. Proteins that had >2 peptides matching the above criteria were considered confirmed assignments while proteins identified with one peptide regardless of the Mascot score were highlighted as tentative assignments. Automated differential quantification of peptides in a set of samples was accomplished with Proteomarker.

### Data Quality Control—Prefiltering, Imputation and Normalization

Subsequent to raw data acquisition and processing, data quality control and prefiltering were done for this study as previously described. Briefly, a three-step pre-filtering was performed to resolve some of the peak mis-alignment issues, and remove those peptides in the abundance matrix that received very poor quality identifications or no qualitative identification at all. A peptide was rejected if (i) its consensus sequence was not assigned at all or (ii) the consensus peptide sequence score was below the 74th percentile of all scores (≦Mascot score of 21.125). Next, intensity summaries of identical sequence peptides were integrated with annotations retained from the diffset with the least number of missing values (including retention time, m/z ratio, sequence, score, and protein annotation). A final pre-filtering procedure was carried out to retain those peptides only for which the observed missing count per peptide was strictly less than 50% per experimental unit while maximizing the total number of peptides remaining after selection. The final number of peptides retained was p=2584 (1,360 proteins).

### Missing Value Imputation

Missing values in LC/MS data arise because of imperfect detection and alignment of peak intensities or by true absence of expression. To account for the non-random nature of the missingness mechanism at play (non-ignorable left-censoring) and its extent in this type of data (non-ignorable left-censoring), we used a probability model adapted from Wang which describes ‘artefactual missing events’. This model makes inferences on the missing values of one sample based on the information from other ‘similar’ samples (technical replicates or nearest neighbors). It substitutes a missing measurement of intensity with its expected value of the true intensity given that it is unobservable. Estimation of the imputation parameters was done in order to minimize the percentage of remaining missing values. The initial number of missing values after the above pre-filtering was 60.8%. Remaining missing values after imputation (42.2%) represent truly absent peptides in the samples and were typically imputed by taking an estimate of the background noise.

### Variance Stabilization of the Data Features

To remove sources of systematic variation due to experimental artifacts in the measured intensities and to ensure that the usual assumptions for statistical inferences are met (normality, homoscedasticity), we applied a variance stabilization and normalization transformation on the variables (peptides). We used the joint adaptive mean-variance regularization procedure. This method overcomes the lack of degrees of freedom and issues with variance-mean dependency common in high-dimensional proteomics datasets where the number of variables dominates the number of samples.

### Statistical Analysis

### Unsupervised Analyses

Potential groups and outliers among the samples were checked by a Principal Component Analysis (PCA). Clustering analysis was performed using complete linkage hierarchical clustering and the gap statistics to estimate the real number of clusters in the data

### Predictive Proteomics Model

Patients were re-labeled at visit 1 with their MA status at visit 3. This new response variable was then regressed onto all peptide expression levels at visit 1 by fitting a generalized (logistic) linear model via penalized maximum likelihood (elastic net regularization). Fitting was carried out by 4-fold cross-validation with the help of the R implementation in the ‘glmnet’ CRAN package. This allowed the selection of peptides predictors as early as visit 1 with best predictive value of progression towards MA at visit 3, as well as the determination of individual probability of MA progression by patient.

### ELISA Verification of Target Urine Biomarkers

Four putative urine biomarkers identified in the label free proteomic analysis as having significant abundance at visit 1 were selected for verification via ELISA as well and additional protein, granulin which was identified in the analysis but not found significant at visit 1. Urine samples collected at visit 1 from a total of 110 study subjects were analyzed. In addition, decline in estimated glomerular filtration rate (eGFR) was examined in patients with type 1 diabetes, and 13 participants with normal urinary albumin excretion and 5 participants who developed albuminuria also experienced a significant decline in eGFR (≧3.3% per year). The five biomarkers were measured by commercially available ELISA kits according to the manufacturer's instructions: Tamms Horsfall glycoprotein (MD Biosciences, St. Paul, Minn.); human progranulin (R&D Systems, Minneapolis, Minn.); human clusterin (BioVendor, Candler, N.C.); human alpha-1-acid glycoprotein (Assaypro, St. Charles, Mo.) and prostaglandin D synthetase (BioVendor, Candler, N.C.). All kits have inter and intra assay coefficients of variation of less than 15%.

### Results

### Label Free Expression Analysis

Characteristics of study participants in the label free expression analysis at the baseline study visit are shown in Table 3. Study participants with type 1 diabetes who developed micro- or macroalbuminuria did not differ from those who remained normoalbuminuric over the six years of the study in terms of age, diabetes duration, sex, HbA1c, or baseline AER. The only significant difference by groups was the significantly increased AER at the end of the study those who developed micro- or macroalbuminuria. The sample preparation protocol was reproducible across individual samples and yielded sufficient protein concentrations with ranges of 0.206 μg/μL to 39 μg/μL. Reproducible protein patterns via 1D-SDS PAGE were observed across all samples (data not shown). These samples were subsequently digested and analyzed by LC/MS/MS as described in the methods section. Distinct chromatographic differences were observed in normoalbuminuric and microalbuminuric samples. The analysis provided 1115 tentative protein assignments (at least 1 peptide sequenced with reproducible chromatographic entities) and 246 confirmed protein assignments (at least 2 or more peptide sequenced).

TABLE 3

| | | Patients with |
|---|---|---|
| | Patients with | type 1 diabetes |
| | type 1 diabetes | who progressed to |
| | and normoalbuminuria | albuminuria |
| | N = 11 | N = 13 |
| Age (years) | 39 ± 9 | 41 ± 8 |
| Diabetes duration (years) | 23 ± 10 | 22 ± 9 |
| Sex (% male) | 55% | 46% |
| HbA1c (%) | 8.1 ± 0.7 | 8.2 ± 1.7 |
| Baseline AER | 6.0 ± 3.0 | 10.5 ± 5.6 |
| Final AER | 5.0 ± 2.3^{a} | 89.0 ± 114.1 |

^{a}P < 0.05 for comparison of patients with type 1diabetes and normaolbuminuria vs. albuminuria

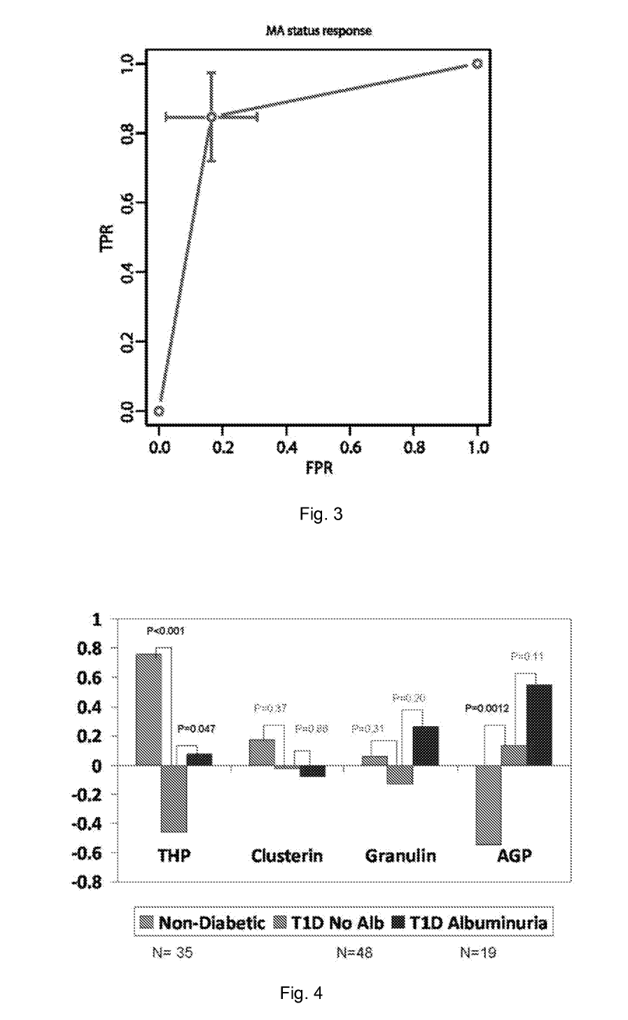

To build a predictive model of the progression of the T1DM and make an early determination of which of the apparently normal patients at visit 1 are likely to progress in the MA disease over the years, we looked at the data in a cross-sectional way and built predictive models at visit 1 using a discrete cutoff for MA status per patient at visit 3. In this analysis, we selected 252 peptide predictors (corresponding to 183 proteins) with the best predictive value of progression towards MA at later visits. This formed the basis of a predictive proteomics model, which determines the individual probability of MA progression by patient, whether the patient has already been observed or is incoming (new). FIG. 3 highlights the predictive model performance. The model yielded an area under the receiver operating curve (AUC) of 84.7% (+/−5.3%) with a true positive rate of 84.7% (+/−12.7) which corresponded to 19 of 22 correctly classified at visit 1. An equal distribution of increasing and decreasing peptide abundances in TIDM patients who progress to MA compared to TIDM patients who did not develop MA was observed. Overall, 148 peptides decreased in abundance in the T1DM MA groups while 104 peptides increased. Table 4 highlights proteins whose peptides were used in the model and for which more than 1 peptide was observed in the label free analysis. The directional change for albumin was consistent between AER and label free albumin measures at visit 1 with values increasing in the T1DM MA group. The median AER value for T1DM and T1DM MA was 5.64 and 9.42 respectively while the label free median peptide intensities (normalized and transformed scale) were 0.67 for T1DM and 0.85 for T1DM MA.

TABLE 4

| | | Numbers of |
|---|---|---|
| | | Peptides |
| Protein ID | Protein Description | Identified |
| IPI00745872 | ALB Isofrom 1 of Serum albumin | 169 |
| IPI00022426 | AMBP Protein AMBP | 42 |
| IPI00006662 | APOD Apolipoprotein D | 8 |
| IPI00166729 | AZGP1 alpha-2-glycoprotein 1, zinc | 25 |
| IPI00011302 | CD59 CD59 glycoprotein | 21 |
| IPI00220117 | CD99 Putative uncharacterized protein CD99 | 19 |
| IPI00795633 | CLU cDNA FLJ57622, highly similar to | 11 |
| | Clusterin | |
| IPI00017601 | CP Ceruloplasmin | 14 |
| IPI00021885 | FGA Isoform 1 of Fibrinogen alpha chain | 10 |
| IPI00024284 | HSPG2 Basement membrane-specific | 8 |
| | heparin sulfate proteoglycan core protein | |
| IPI00472610 | IGHM IGHM protein | 25 |
| IPI00382938 | IGLV4-3 IGLV4-3 protein | 19 |
| IPI00215894 | KNG1 Isoform LMW of Kininogen-1 | 23 |
| IPI00220327 | KRT1 Keratin, type II cytoskeletal 1 | 10 |
| IPI00019359 | KRT9 Keratin, type I cytoskeletal 9 | 7 |
| IPI00028714 | MGP Matrix Gla protein | 6 |
| IPI00301579 | NPC2 cDNA FLJ59142, highly similar to | 4 |
| | Epididymal secretory protein E1 | |
| IPI00022429 | ORM1 Alpha-1-acid glycoprotein1 | 19 |
| IPI00022974 | PIP Prolactin-inducible protein | 4 |
| IPI00219825 | PSAP Prosaposin | 12 |
| IPI00013179 | PTGDS Prostaglandin-H2 D-isomerase | 25 |
| IPI00023020 | SEMG1 Isoform 1 of Semenogelin-1 | 61 |
| IPI00025415 | SEMG2 Semenogelin-2 | 30 |
| IPI00553177 | SERPINA1 Isoform 1 of Alpha-1-antitryspin | 19 |
| IPI00032179 | SERPINC1 Antithrombin III variant | 2 |
| IPI00291866 | SERPING1 Plasma protease C1 inhibitor | 11 |
| IPI00477896 | SLC26A6 Anchor protein | 2 |
| IPI00022620 | SLURP1 Secreted Ly-6/uPAR-related | 8 |
| | protein 1 | |
| IPI00303112 | TET1 Protein TET1 | 2 |
| IPI00022463 | TF Serotransferrin | 48 |
| IPI00013945 | UMOD Isoform 1 of Uromodulin | 62 |
| IPI00289501 | VGF Neurosecretory VGF | 3 |

### ELISA Protein Analysis

Characteristics of study participants at the baseline exam were examined between non-diabetic control participants (Group A), patients with type 1 diabetes who remained normoalbuminuric and had normal renal function (Group B), patients with type 1 diabetes who developed renal function decline without albuminuria (Group C) and patients with type 1 diabetes who went on to develop albuminuria (Group D) (Table 5). Non-diabetic controls (Group A) were significantly older than type 1 diabetes patients with normal renal function and normoalbuminuria (Group B), and participants in Group B were significantly younger than those who developed albuminuria (Group D). There were no differences in diabetes duration or sex. HbA1c was significantly lower in Group B compared to both Groups C and D. Baseline AER and eGFR did not differ between non-diabetic controls (Group A) and type 1 diabetes patients with no renal decline and normoalbuminuria (Group B), but baseline AER was significantly lower in Group A compared to Groups C and D, and in Groups B and C compared to Group D.

TABLE 5

| | | Type 1 | | |
|---|---|---|---|---|
| | | diabetes | Type 1 | Type 1 |
| | | patients with | diabetes | diabetes |
| | | normal renal | patients | patients who |
| | | function | with renal | progressed |
| | Non-Diabetic | and no | function | to |
| | controls | albuminuria | decline | albuminuria |
| | Group A | Group B | Group C | Group D |
| | N = 35 | N = 35 | N = 15 | N = 19 |
| Age (years) | 39 ± 9^{a} | 33 ± 9^{d} | 35 ± 10 | 39 ± 8 |
| Diabetes | N/A | 19 ± 7 | 23 ± 10 | 22 ± 8 |
| duration | | | | |
| (years) | | | | |
| Sex | 46 | 50 | 39 | 47 |
| (% male) | | | | |
| HbA1c (%) | 5.4 ± 0.4^{a,b,c} | 7.6 ± 0.9^{d,e} | 8.9 ± 1.9 | 8.5 ± 1.4 |
| Baseline | 3.8 ± 1.3^{b,c} | 5.2 ± 3.3^{e} | 7.0 ± 4.2^{f} | 10.3 ± 5.0 |
| AER | | | | |
| Final AER | 3.8 ± 1.2^{c} | 5.0 ± 2.8^{e} | 5.7 ± 4.3^{f} | 171 ± 430 |
| Cystatin C | 0.78 ± 0.07 | 0.79 ± 0.09 | 0.74 ± 0.17 | 0.80 ± 0.12 |
| Baseline | 130 ± 13^{b} | 129 ± 16^{e} | 142 ± 29^{f} | 128 ± 20 |
| eGFR | | | | |
| Final e GFR | 134 ± 11^{b,c} | 135 ± 16^{d,e} | 118 ± 25 | 121 ± 29 |
| Uric acid | 5.4 ± 1.04 | 4.8 ± 0.88 | 5.0 ± 1.3 | 5.4 ± 1.3 |

^{a}p < 0.05 Group A vs. Group B
^{b}p < 0.05 Group A vs. Group C
^{c}p < 0.05 Group A vs. Group D
^{d}p < 0.05 Group B vs. Group C
^{e}p < 0.05 Group B vs. Group D
^{f}p < 0.05 Group C vs. Group D

Levels of Tamms Horsfall Protein (THP), progranulin, clusterin, and alpha-1-acid glycoprotein (AGP) were compared between Groups A, B, C and D (Table 6). THP was significantly higher in Group A compared to all other groups, and was lower in Group B compared to Group D. AGP was significantly lower in Group A compared to all other groups, and in Group B compared to both Groups C and D.

TABLE 6

| | | | Type 1 diabetes | | Type 1 diabetes | | Type 1 diabetes | |
|---|---|---|---|---|---|---|---|---|
| | | | patients with normal | | patients with | | patients who | |
| | Non-Diabetic | | renal function and | | renal function | | progressed to | |
| | controls | | normoalbuminuria | | decline | | albuminuria | |
| | Group A | | Group B | | Group C | | Group D | |
| | N = 35 | | N = 34 | | N = 15 | | N = 19 | |
| THP | 16,500^{a,b,c} | (2,254-101,000) | 2,619^{e} | (86-26,700) | 3,443 | (52-24,800) | 4,251 | (707-35,500) |
| Progranulin | 3.26 | (.078-10.64) | 1.95 | (0-18.60) | 3.59 | (0-24.46) | 3.83 | (0.66-23.63) |
| Clusterin | 0.6 | (0-27.3) | .01 | (0-12.3) | 0 | (0-2.3) | 0 | (0-10.9) |
| AGP | 118^{a,b,c} | (14-1025) | 267^{d,e} | (27-1829) | 481 | (31-4549) | 0 | (64-3292) |

^{a}p < 0.05 Group A vs. Group B
^{b}p < 0.05 Group A vs. Group C
^{c}p < 0.05 Group A vs. Group D
^{d}p < 0.05 Group B vs. Group C
^{e}p < 0.05 Group B vs. Group D

Standardized z-scores were calculated for all four proteins and were examined among non-diabetic controls, type 1 diabetes patients with normal urinary albumin levels and type 1 diabetes patients who progressed to albuminuria (FIG. 3). THP levels were significantly lower in normoalbuminuric patients with type 1 diabetes compared to normal controls, and were significantly higher in patients with type 1 diabetes who progressed to albuminuria compared to normoalbuminuric patients with type 1 diabetes. AGP levels were significantly lower in non-diabetic controls compared to patients with type 1 diabetes and normoalbuminuria. In multivariable ordinal logistic regression modeling, THP, progranulin and AGP were significantly predictive of renal decline and albuminuria in patients with type 1 diabetes, adjusting for age, diabetes duration, HbA1c, baseline AER, uric acid and cystatin C (Table 7).

TABLE 7

| | OR (95% CI) | P-Value | C-statistic |
|---|---|---|---|
| THP | 2.2 | 0.03 | 0.862 |
| | (1.1-4.5) | | |
| Clusterin | 1.6 | 0.11 | 0.832 |
| | (0.9-2.8) | | |
| Granulin | 31 | 0.002 | 0.898 |
| | (1.5-6.4) | | |
| AGP | 2.2 | 0.02 | 0.862 |
| | (1.1-4.9) | | |

All models were adjusted for age, diabetes duration HbA1c, AER, uric acid and cystatin C

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

1. A method for diagnosing a renal disorder in a subject, said method comprising the steps of:
obtaining a biological sample from the subject; and
determining, in the biological sample, a level of one or more of the polypeptide selected from the group consisting of Isoform 1 of Uromodulin, Serotransferrin precursor, Isoform Long of Trifunctional purine biosynthetic protein adenosine-3, Cystatin-A, alpha-2-glycoprotein 1 zinc, ubiquitin and ribosomal protein S27a precursor, C16orf73 Novel protein, Zinc finger homeobox protein 2, Muscarinic acetylcholine receptor M1, WDR2OWD repeat domain 20 isoform 3, Interleukin 20 receptor alpha, Isoform 4 of VPS10 domain-containing receptor SorCS1 precursor, CDNA FLI41726 fis clone HLUNG2014449, Similar to CF3558-PA isoform A, Solute carrier family 16 member 2, trefoil factor 3 precursor, RNASE2 Nonsecretory ribonuclease precursor, Secreted Ly-6/upar-related protein 1 precursor, RGSL1 Hypothetical Protein, Isoform LMW of Kininogen-1 precursor, Isoform 3 of Killer cell immunoglobulin-like receptor 2DL4 precursor, Melanoma-associated antigen B6, isoform A of AP-2 complex subunit alpha-1, secreted phosphoprotein 1 isoform b, Cytochrome P450 4F11, Insulin growth factor-like family member 4 precursor, SLC39A7 Non-class II protein, C19orf57 Uncharacterized protein, SERPINA Alpha-1-antitrypsin precursor, ALB Isoform 1 of Serum albumin precursor, and Mucin-5B precursor, wherein an increase or decrease in the level of one or more of the polypeptide compared to a control level is indicative of a renal disorder.

2. The method of claim 1, the renal disorder comprising type 1 diabetes, diabetic nephropathy or end stage renal disease.

3. The method of claim 1, the biological sample comprising a sample of urine.

4. The method of claim 1, wherein the subject is a human being.

5. The method of claim 1, wherein a decreased level of one or more of the polypeptides selected from the group consisting of Isoform 1 of Uromodulin, Serotransferrin precursor, Isoform Long of Trifunctional purine biosynthetic protein adenosine-3, Cystatin-A, alpha-2-glycoprotein 1 zinc, ubiquitin and ribosomal protein S27a precursor, C16orf73 Novel protein, Zinc finger homeobox protein 2, Muscarinic acetylcholine receptor Ml, WDR2OWD repeat domain 20 isoform 3, Interleukin 20 receptor alpha, Isoform 4 of VPS10 domain-containing receptor SorCS1 precursor, CDNA FLI41726 fis clone HLUNG2014449, and Similar to CF3558-PA isoform A compared to a control level is indicative of a renal disorder.

6. The method of claim 1, wherein an increased level of one or more of the polypeptides selected from the group consisting of Solute carrier family 16 member 2, trefoil factor 3 precursor, RNASE2 Nonsecretory ribonuclease precursor, Secreted Ly-6/upar-related protein 1 precursor, RGSL1 Hypothetical Protein, Isoform LMW of Kininogen-1 precursor, Isoform 3 of Killer cell immunoglobulin-like receptor 2DL4 precursor, Melanoma-associated antigen B6, isoform A of AP-2 complex subunit alpha-1, secreted phosphoprotein 1 isoform b, Cytochrome P450 4F11, Insulin growth factor-like family member 4 precursor, SLC39A7 Non-class II protein, C19orf57 Uncharacterized protein, SERPINA Alpha-1-antitrypsin precursor, ALB Isoform 1 of Serum albumin precursor, and Mucin-5B precursor compared to a control level is indicative of a renal disorder.

7. A method for diagnosing type 1 diabetes in a subject, said method comprising steps of:
obtaining a biological sample from the subject; and
determining, in the biological sample, a level of one or more of polypeptide selected from the group consisting of Isoform 1 of Uromodulin, Serotransferrin precursor, Isoform Long of Trifunctional purine biosynthetic protein adenosine-3, Cystatin-A, alpha-2-glycoprotein 1 zinc, ubiquitin and ribosomal protein S27a precursor, C16orf73 Novel protein, Zinc finger homeobox protein 2, Muscarinic acetylcholine receptor M1, WDR2OWD repeat domain 20 isoform 3, Interleukin 20 receptor alpha, Isoform 4 of VPS10 domain-containing receptor SorCS1 precursor, CDNA FLI41726 fis clone HLUNG2014449, Similar to CF3558-PA isoform A, Solute carrier family 16 member 2, trefoil factor 3 precursor, RNASE2 Nonsecretory ribonuclease precursor, Secreted Ly-6/upar-related protein 1 precursor, RGSL1 Hypothetical Protein, Isoform LMW of Kininogen-1 precursor, Isoform 3 of Killer cell immunoglobulin-like receptor 2DL4 precursor, Melanoma-associated antigen B6, isoform A of AP-2 complex subunit alpha-1, secreted phosphoprotein 1 isoform b, Cytochrome P450 4F11, Insulin growth factor-like family member 4 precursor, SLC39A7 Non-class II protein, C19orf57 Uncharacterized protein, SERPINA Alpha-1-antitrypsin precursor, ALB Isoform 1 of Serum albumin precursor, and Mucin-5B precursor, wherein an increase or decrease in the level of one or more of the proteins compared to a control level is indicative of type 1 diabetes.

8. The method of claim 7, wherein the biological sample comprises a sample of urine.

9. The method of claim 7, wherein the subject is a human being.

10. The method of claim 7, wherein a decreased level of one or more of the polypeptides selected from the group consisting of Isoform 1 of Uromodulin, Serotransferrin precursor, Isoform Long of Trifunctional purine biosynthetic protein adenosine-3, Cystatin-A, alpha-2-glycoprotein 1 zinc, ubiquitin and ribosomal protein S27a precursor, C16orf73 Novel protein, Zinc finger homeobox protein 2, Muscarinic acetylcholine receptor M1, WDR20WD repeat domain 20 isoform 3, Interleukin 20 receptor alpha, Isoform 4 of VPS10 domain-containing receptor SorCS1 precursor, CDNA FLI41726 fis clone HLUNG2014449, and Similar to CF3558-PA isoform A compared to a control level is indicative of type 1 diabetes.

11. The method of claim 7, wherein an increased level of one or more of the polypeptides selected from the group consisting of Solute carrier family 16 member 2, trefoil factor 3 precursor, RNASE2 Nonsecretory ribonuclease precursor, Secreted Ly-6/upar-related protein 1 precursor, RGSL1 Hypothetical Protein, Isoform LMW of Kininogen-1 precursor, Isoform 3 of Killer cell immunoglobulin-like receptor 2DL4 precursor, Melanoma-associated antigen B6, isoform A of AP-2 complex subunit alpha-1, secreted phosphoprotein 1 isoform b, Cytochrome P450 4F11, Insulin growth factor-like family member 4 precursor, SLC39A7 Non-class II protein, C19orf57 Uncharacterized protein, SERPINA Alpha-1-antitrypsin precursor, ALB Isoform 1 of Serum albumin precursor, and Mucin-5B precursor compared to a control level is indicative of type 1 diabetes.

12. A method for diagnosing type 1 diabetes responsive to treatment by an angiotensin-converting enzyme inhibitor and/or an angiotensin receptor blocker in a subject, said method comprising steps of:
obtaining a biological sample from the subject; and
determining, in the biological sample, a level of one or more of polypeptide selected from the group consisting of Apolipoprotein D precursor, APOH Beta-2-glycoprotein 1 precursor, CD59 glycoprotein 1 precursor, CD99 Isoform II of CD99 antigen precursor, CD99L2 protein DKFZp761 H2024, CLU, Collagen alpha-1(l) chain precursor, Cystatin-A, Beta-defensin 1 precursor, Isoform 2 of Granulins precursor, Basement membrane-specific heparin sulfate proteoglycan core protein precursor, IGKC protein, Inter-alpha-trypsin inhibitor heavy chain H2 precursor, Isoform LMW of Kininogen-1 precursor, Isoform 1 of Peptidase inhibitor 16 precursor, Polymeric-immunoglobulin receptor precursor, Isoform 2 of Phosphoinositide-3-kinase-interacting protein 1 precursor, isoform Sap-mu-0 of Proactivator polypeptide precursor, Prostaglandin-H2 D-isomerase precursor, Transcriptional activator protein Pur-Alpha, RNASE1 Ribonuclease pancreatic precursor, RNASE2 Nonsecretory ribonuclease precursor, RPS27A;UBC;UBB ubiquitin and ribosomal protein S27a precursor, Secreted Ly-6/uPar-related protein 1 precursor, secreted phosphoprotein 1 isoform b, Trefoil factor 2 precursor, Isoform 1 of Uromodulin precursor, VGF nerve growth factor inducible precursor, Isoform 1 of WAP four-disulfide core domain protein 2 precursor, AMBP protein precursor, Annexin A1, alpha-2-glycoprotein 1 zinc, Beta-2-microglobulin precursor, C gamma 3, Ceruloplasmin precursor, Hemopexin precursor, Mucin-5B precursor, ORM2 Alpha-1-acid glycoprotein 2 precursor, SERPINA1 Alpha-1-antitrypsin precursor, and TF Serotransferrin precursor, wherein an increase or decrease in the level of one or more of the proteins compared to a control level is indicative of a sub-type of type 1 diabetes responsive to treatment by an angiotensin-converting enzyme inhibitor and/or an angiotensin receptor blocker.

13. The method of claim 12, wherein the biological sample comprises a sample of urine.

14. The method of claim 12, wherein the subject is a human being.

15. The method of claim 12, wherein a decreased level of one or more of the polypeptides selected from the group consisting of Apolipoprotein D precursor, APOH Beta-2-glycoprotein 1 precursor, CD59 glycoprotein 1 precursor, CD99 Isoform II of CD99 antigen precursor, CD99L2 protein DKFZp761 H2024, CLU, Collagen alpha-1(I) chain precursor, Cystatin-A, Beta-defensin 1 precursor, Isoform 2 of Granulins precursor, Basement membrane-specific heparin sulfate proteoglycan core protein precursor, IGKC protein, Inter-alpha-trypsin inhibitor heavy chain H2 precursor, Isoform LMW of Kininogen-1 precursor, Isoform 1 of Peptidase inhibitor 16 precursor, Polymeric-immunoglobulin receptor precursor, Isoform 2 of Phosphoinositide-3-kinase-interacting protein 1 precursor, isoform Sap-mu-0 of Proactivator polypeptide precursor, Prostaglandin-H2 D-isomerase precursor, Transcriptional activator protein Pur-Alpha, RNASE1 Ribonuclease pancreatic precursor, RNASE2 Nonsecretory ribonuclease precursor, RPS27A;UBC;UBB ubiquitin and ribosomal protein S27a precursor, Secreted Ly-6/uPar-related protein 1 precursor, secreted phosphoprotein 1 isoform b, Trefoil factor 2 precursor, Isoform 1 of Uromodulin precursor, VGF nerve growth factor inducible precursor, Isoform 1 of WAP four-disulfide core domain protein 2 precursor compared to a control level is indicative of a sub-type of type 1 diabetes responsive to treatment by an angiotensin-converting enzyme inhibitor and/or an angiotensin receptor blocker.

16. The method of claim 12, wherein an increased level of one or more of the polypeptides selected from the group consisting of AMBP protein precursor, Annexin A1, alpha-2-glycoprotein 1 zinc, Beta-2-microglobulin precursor, C gamma 3, Ceruloplasmin precursor, Hemopexin precursor, Mucin-5B precursor, ORM2 Alpha-1-acid glycoprotein 2 precursor, SERPINA1 Alpha-1-antitrypsin precursor, and TF Serotransferrin precursor compared to a control level is indicative of a sub-type of type 1 diabetes responsive to treatment by an angiotensin-converting enzyme inhibitor and/or an angiotensin receptor blocker.

17-21. (canceled)

22. A diabetes expression profile map comprising expression level information for one or more of polypeptides selected from the group consisting of Isoform 1 of Uromodulin, Serotransferrin precursor, Isoform Long of Trifunctional purine biosynthetic protein adenosine-3, Cystatin-A, alpha-2-glycoprotein 1 zinc, ubiquitin and ribosomal protein S27a precursor, C16orf73 Novel protein, Zinc finger homeobox protein 2, Muscarinic acetylcholine receptor M1, WDR2OWD repeat domain 20 isoform 3, Interleukin 20 receptor alpha, Isoform 4 of VPS10 domain-containing receptor SorCS1 precursor, CDNA FLI41726 fis clone HLUNG2014449, Similar to CF3558-PA isoform A, Solute carrier family 16 member 2, trefoil factor 3 precursor, RNASE2 Nonsecretory ribonuclease precursor, Secreted Ly-6/upar-related protein 1 precursor, RGSL1 Hypothetical Protein, Isoform LMW of Kininogen-1 precursor, Isoform 3 of Killer cell immunoglobulin-like receptor 2DL4 precursor, Melanoma-associated antigen B6, isoform A of AP-2 complex subunit alpha-1, secreted phosphoprotein 1 isoform b, Cytochrome P450 4F11, Insulin growth factor-like family member 4 precursor, SLC39A7 Non-class II protein, C19orf57 Uncharacterized protein, SERPINA Alpha-1-antitrypsin precursor, ALB Isoform 1 of Serum albumin precursor, and Mucin-5B precursor, analogs and fragments thereof.

23. The diabetes expression profile map of claim 22, wherein the expression profile map comprises expression level information for at least one biological sample obtained from a subject with type 1 diabetes or an individual without type 1 diabetes.

24. The diabetes expression profile map of claim 22, wherein the biological sample comprises a sample of urine.

25. A diabetes expression profile map comprising expression level information for one or more of polypeptides selected from the group consisting of Apolipoprotein D precursor, APOH Beta-2-glycoprotein 1 precursor, CD59 glycoprotein 1 precursor, CD99 Isoform II of CD99 antigen precursor, CD99L2 protein DKFZp761 H2024, CLU, Collagen alpha-1(I) chain precursor, Cystatin-A, Beta-defensin 1 precursor, Isoform 2 of Granulins precursor, Basement membrane-specific heparin sulfate proteoglycan core protein precursor, IGKC protein, Inter-alpha-trypsin inhibitor heavy chain H2 precursor, Isoform LMW of Kininogen-1 precursor, Isoform 1 of Peptidase inhibitor 16 precursor, Polymeric-immunoglobulin receptor precursor, Isoform 2 of Phosphoinositide-3-kinase-interacting protein 1 precursor, isoform Sap-mu-0 of Proactivator polypeptide precursor, Prostaglandin-H2 D-isomerase precursor, Transcriptional activator protein Pur-Alpha, RNASE1 Ribonuclease pancreatic precursor, RNASE2 Nonsecretory ribonuclease precursor, RPS27A;UBC;UBB ubiquitin and ribosomal protein S27a precursor, Secreted Ly-6/uPar-related protein 1 precursor, secreted phosphoprotein 1 isoform b, Trefoil factor 2 precursor, Isoform 1 of Uromodulin precursor, VGF nerve growth factor inducible precursor, Isoform 1 of WAP four-disulfide core domain protein 2 precursor, AMBP protein precursor, Annexin A1, alpha-2-glycoprotein 1 zinc, Beta-2-microglobulin precursor, C gamma 3, Ceruloplasmin precursor, Hemopexin precursor, Mucin-5B precursor, ORM2 Alpha-1-acid glycoprotein 2 precursor, SERPINA1 Alpha-1-antitrypsin precursor, and TF Serotransferrin precursor, analogs and fragments thereof.

26. The diabetes expression profile map of claim 25, wherein the expression profile map comprises expression level information for at least one biological sample obtained from a subject with type 1 diabetes or an individual with a sub-type of type 1 diabetes responsive to treatment by an angiotensin-converting enzyme inhibitor and/or an angiotensin receptor blocker.

27. The diabetes expression profile map of claim 25, wherein the biological sample comprises a sample of urine.

28-37. (canceled)
